(19) United States
(12) Patent Application Publication
(10) Pub. No.: US 2014/0228234 A1
(43) Pub. Date: Aug. 14, 2014

(54) TARGET-RECOGNITION COMPOSITIONS COMPRISING NOVEL SYNTHETIC CONJUGATES FOR TRAPPING AND DIAGNOSIS OF A TARGET MOLECULE

(71) Applicant: Naomi B. Zak, Jerusalem (IL)

(72) Inventor: Naomi B. Zak, Jerusalem (IL)

(21) Appl. No.: 14/126,088

(22) PCT Filed: Jun. 14, 2012
(86) PCT No.: PCT/IL2012/000236

§ 371 Date: Mar. 20, 2014

Related U.S. Application Data
(60) Provisional application No. 61/497,077, filed on Jun. 15, 2011.

(51) Int. Cl. G01N 33/86; G01N 33/543

(52) U.S. Cl. CPC G01N 33/86; G01N 33/54306
USPC 506/9; 436/501; 422/69; 435/6.1; 435/287.2

(57) ABSTRACT

The invention relates to methods, kits and devices for sensitive and efficient trapping, removing, detecting or quantification of a target compound in or from a substance. More specifically, the invention encompasses the use of at least one target-recognition composition comprising at least one nucleic acid-based aptamer that specifically recognizes and binds the target compound, and at least one DNA dendrimer labeled with at least one trapping, partitioning or detectable moiety.

### FIELD OF THE INVENTION

The present invention relates to methods for increasing the sensitivity of trapping, removal, detection or quantification of a target compound present in a substance. More particularly, the invention provides sensitized methods, target-recognition compositions and devices utilizing aptamer molecules as targeting elements and dendrimers as partitioning or detecting elements providing efficient and improved removal, trapping, detection or quantification of specific targets.

### BACKGROUND OF THE INVENTION

Throughout this application, various publications, including United States patents, are referenced by author and year, or by number, respectively. The disclosures of these publications and patents and patent applications in their entireties are hereby incorporated by reference into this application in order to more fully describe the state of the art to which this invention pertains.

Single stranded DNA or RNA molecules with highly specific binding to target molecules are referred to as nucleic acid ligands or aptamers. These are obtained from random sequence oligonucleotide libraries by exposure to a target of interest and selection of binding molecules. Aptamers bind specifically and with high affinity to a desired target compound or molecule through electrostatic, hydrogen bonding and hydrophobic interactions that also govern the affinity and specificity in the formation of antibody-antigen complexes. Indeed, affinities of nucleic acid ligands to their targets often lie in the same range observed for the structurally much larger antibody-antigen complexes.

Analytical applications that allow molecular detection, quantification and partitioning of contaminating analytes as well as molecular imaging technologies are important tools in the determination of food quality, in environmental safety testing, in medical diagnostics and in research. These applications are constantly evolving to improve their performance, as well as to adapt to new analytes. These applications encompass, inter alia, molecules that recognize other molecules with high specificity and affinity. Examples of such molecules, which are important for a wide range of applications, are antibodies and nucleic acids that are used as probes for binding to complementary oligonucleotide strands through Watson-Crick base pairing. The discovery of aptamers as nucleic acid ligands that have specific high affinity binding to non-nucleic acid or nucleic acid molecules through interactions other than classic Watson-Crick base pairing [Tuerk C. and Gold L. Science (1990) 249: 505-510] offers a new molecular class with the potential to fulfill the roles played by antibodies.

The therapeutic use of aptamers is currently well established, however, the potential of aptamers in various analytical applications, such as the detection of molecules in safety testing of food and environment and in medical diagnostics has not been fully realized and these areas are still dominated by the use of antibodies [Tombelli S. et al., Biomol Eng., (2007) June 24(2):191-200]. Nonetheless, several aptamer-based assays with very good sensitivity have been proposed for cytokines and growth factors, which are of particular importance in clinical and medical research.

Direct comparisons of aptamers and antibodies which are specific for the same molecule (e.g., IgE or HIV-1 Tat) used as bio-recognition elements in biosensors or biochips [Minunni M. et al., Biosens. Bioelectron. (2004) December 15; 20(6):1149-56] reported similar sensitivities and reproducibility for both receptors. However, because of the usual range of aptamer affinities for their targets in the nanomolar or sub-nanomolar level, the very high sensitivities (at the pM concentrations range) required for aptamer-based assays sometimes cannot be reached by simple “direct” binding protocols. Consequently, recent efforts are focused on improving the analytical detection limits of assays relying on the use of aptamers as capturing agents/receptors. For example, the aptamer-based assay may be coupled to different amplification strategies. Several methodologies have been successfully employed as signal amplification tools, such as metallic and magnetic particles, enzymatic labels or quantum dots. Other amplification techniques include polymerase chain reaction or combination of DNA/RNA catalytic molecules with aptamers. However, from a commercial point of view, all of these strategies suffer from a similar drawback in that they involve a large departure from the currently used ELISA and other antibody-based assays (such as competition assays) and thus present a large barrier to acceptance in existing diagnostic laboratory settings with established working routines. In addition, these methodologies cannot easily be incorporated into imaging protocols to increase the sensitivity of detection. Thus, there is a need for a universally applicable and methodologically simple signal amplification procedure that will increase the sensitivity of aptamer based detection.

Molecules that provide signal amplification in various binding assays, by virtue of the fact that they contain multiple labels or attachment sites for label moieties, are known in the art and include nucleic acid based and other synthetic dendritic structures, hydrocarbon polymers, proteins and other types of molecules. Inclusion of multiply labeled polymers as a component of a detection assay comprising antibodies or nucleic acid probes (that bind to complementary nucleic acid targets) has been shown to increase the sensitivity of the detection by several orders of magnitude.

Among the highly labeled polymeric molecules available, dendrimers comprised of nucleic acids offer many advantages and great versatility for combination with nucleic acid aptamers, in assays intended to detect one or more targets. The combined use of recognition molecules (aptamers) and signal amplification molecules (dendrimers) of the same molecular species, as shown for the first time by the present invention, enables additional simple and easily controlled approaches to the generation of highly labeled recognition complexes. Nucleic acid dendrimers can be directly modified through the covalent attachment of fluorophores or other signal molecules. In the case of a single target, for example, a biotinylated aptamer can be used in conjunction with an avidin-bearing dendrimer that is also highly labeled with a fluorophore, in order to amplify the signal production. In the case of multiple targets, specific aptamer binding to fluorophore-specific dendrimers can be effectuated in a similar manner to the above for each aptamer, prior to exposure to the target, and then all aptamer-dendrimer moieties may be mixed.

Although not exemplified, therapeutic uses of DNA dendrimers-antibody conjugates labeled with radiation absorbing nanoparticles were recently suggested as thermal ablation device specific for tumor cells (WO 2011/106481). Still further, a Hybrid DNA Aptamer-Dendrimer Nanomaterial for Targeted Cell Labeling was previously disclosed [Zhou J. et al., Macromol. Biosci. (2009) 9:831-835]. The aptamer-dendrimer disclosed in Zhou J. et al comprises a poly-amidoamine polymer (PAMAM) dendrimer and cancer treatment is the ultimate aim. However, despite the potential signal amplification that can be provided by dendrimers, this publication does not demonstrate an increased sensitivity as a result of the use of dendrimers. It should be emphasized that the dendrimer used in the aptamer-dendrimer disclosed by Zhou et al. was a poly-amidoamine polymer (PAMAM) and lacked key advantages that a nucleic acid dendrimer offers when combined with a nucleic acid aptamer, as instantly claimed. The advantages offered by the present aptamer-dendrimer combination, but missing from the combination disclosed by Zhou et al. include simplicity and versatility. While attachment of DNA aptamers to PAMAM dendrimers is possible, it requires modification of aptamers by addition of carbon molecules and an amino group, activation of carboxy groups on the PAMAM and formation of an amide bond, whereas the DNA aptamer does not require these modifications and may be attached by a variety of well known, and comparatively cheap methods, such as ligation, hybridization with or without cross-linking, and biotinylation. The simplicity of attachment and the option to select specific DNA sequences both in the aptamer and dendrimer facilitate a controlled approach to attachment, for example of multiple aptamers, and especially attachment of different aptamers to a single dendrimer. It is not overlooked that the ability to control the number and the type of aptamers which attach to dendrimers, including attaching different kinds of aptamers to single dendrimers, allows enhanced binding to molecular targets through multivalent interactions with different target epitopes. This is especially advantageous for trapping/inactivation of harmful contaminants.

Moreover, the ability to change the number of aptamers attached to each dendrimer provides the capacity for fine-tuning of molecular target binding sensitivity and specificity.

It should be noted that different objects of the invention will become apparent as the description proceeds.

### SUMMARY OF THE INVENTION

In the first aspect, the present invention provides a method for trapping, removing, detecting or quantitating, at least one target compound in or from a substance or any preparation or sample thereof. In a first step (a), the method involves providing at least one target-recognition composition comprising at least one nucleic acid-based aptamer that specifically recognizes and binds the at least one target compound, or complex thereof, or any parts or fragments thereof, or any combinations thereof, and at least one nucleic acid based dendrimer labeled with at least one trapping and/or partitioning and/or detectable moiety.

The next step (b) involves contacting the substance, or any preparation or a sample, or an aliquot thereof with at least one of the target-recognition composition of (a) under conditions suitable for recognition and binding of the at least one target compound or any parts or fragments thereof, or any combinations thereof, by the at least one nucleic acid based aptamer to form at least one target compound-target recognition composition complex.

The final step (c), involves trapping or detecting the at least one detectable or trapping moiety in the labeled dendrimer comprised within the target-recognition composition, which dendrimer is bound to or conjugated with the at least one aptamer in the target compound-target-recognition composition complex, thereby trapping, removing or determining the presence and optionally, the amount of said target compound in or from said substance.

In the second aspect, the present invention relates to a kit for trapping, removing, detecting, or quantification of at least one target compound in/or from a substance or any preparation or sample thereof. In certain embodiments, the kit of the invention may comprise:

(a) at least one nucleic acid based aptamer that specifically recognizes and binds said at least one target compound, or complex thereof, or any parts or fragments thereof, or any combinations thereof; and (b) at least one nucleic acid based dendrimer labeled with at least one trapping, partitioning or detectable moiety.

In an optional embodiment, the kit of the invention may further comprise at least one capture composition comprising at least one nucleic acid based or amino-acid-based recognition element that specifically recognizes and binds at least one of said target compound or any parts or fragments thereof, or any combinations thereof, said at least one capture-composition is attached to a solid support or a partitioning element.

In a third aspect, the invention relates to a device for trapping, removing, detecting or quantitating, at least one target compound in/or from a substance or any preparation or sample thereof. In certain embodiments, the device of the invention may comprise:

(a) at least one target-recognition composition comprising at least one nucleic acid based aptamer that specifically recognizes and binds the at least one target compound, or complex thereof, or any parts or fragments thereof, or any combinations thereof, and at least one nucleic acid based dendrimer labeled with at least one trapping, partitioning and/or detectable moiety; and
(b) a solid support suitable for the reception and transport of the sample.

These and other aspects of the invention will become apparent by the hand of the following drawings.

### BRIEF DESCRIPTION OF THE FIGURES

FIG. 1

### Conjugation of Aptamer B to Dendrimer Enhances Signal to Background Ratio

The biotinylated aptamer B and the seven different biotinylated aptamer-dendrimer conjugates shown in Tables 1 and 5 (at 50 ng/well) were applied to duplicate wells that had been exposed to different thrombin concentrations (0-1000 ng). Following incubation (2 hrs.), the wells containing attached biotinylated aptamer and biotinylated aptamer-dendrimer complexes were washed and binding of aptamer-dendrimer conjugates to thrombin was determined.

Abbreviations: B (aptamer serial number) (number of dendrimer layers) nB (number of aptamers per dendrimer) Bio (Number of labels per dendrimer), sig. (signal), Bkd (background) Rat. (ratio), Thromb (thrombin amount in ng).

FIG. 2

### Conjugation of Aptamer B to Dendrimer Enhances Signal to Background Ratio at Lower Thrombin Concentrations

The biotinylated aptamer B and the seven different biotinylated aptamer-dendrimer conjugates shown in Tables 1 and 5 (at 100 ng/well) were applied to duplicate wells that had been exposed to different thrombin concentrations (0-250 ng). Following incubation (3 hrs), the wells containing attached biotinylated aptamer and biotinylated aptamer-dendrimer complexes were washed and binding of aptamer-dendrimer conjugates to thrombin was determined.

Abbreviations: B (aptamer serial number), sig. (signal), Bkd (background) Rat. (ratio), Thromb (thrombin amount in ng).

FIG. 3

### SDS Gel Analysis of Thrombin Trapped by Targeting Molecules

An SDS gel is presented, in which A0 denotes the aptamer (A0) and A1, A4, A5, A6 denotes aptamer-dendrimer conjugates (A1, A4, A5 and -A6) used to trap thrombin from solution. The left lane presents markers (25 and 37 kDa) and “Thromb” denotes thrombin, as a control.

### DETAILED DESCRIPTION OF THE INVENTION

Described herein are methods, compositions and devices for increasing the efficiency of trapping, partitioning and removal of a target compound from a substance as well as increasing the sensitivity of detection and quantification of said target compound by a nucleic acid ligand recognition aptamer molecule through conjugation or attachment of said aptamer with a multiply-labeled nucleic acid dendrimer. The diagnostic and other applications of this invention include novel aptamer-based microtitire, lateral flow or flow through assays employing high affinity nucleic acid ligands, specifically, aptamers, as targeting components and polymeric molecules called dendrimers that according to certain embodiments may be labeled and serve as trapping or detection components. Together, the aptamers and polymeric labeled dendrimers comprise target-recognition compositions. The invention enables efficient removal or trapping of target analytes from all substances including food matrices, water, industrial wastes, biological fluids, cell culture media, etc. and in alternative embodiments, further provides sensitive detection and determination of the target quantity found in the substance.

As used herein, a “nucleic acid ligand” is a non-naturally occurring nucleic acid, often referred to as an “aptamer” with specific binding affinity for a target molecule. Nucleic acid aptamers applicable for the present invention may be engineered and selected through any technique or procedure known in the art, for example, repeated rounds of in vitro selection or systematic evolution of ligands by exponential enrichment (SELEX) to bind to various molecular targets, such as small molecules, peptides or proteins, nucleic acids, or any variations thereof to name but few, or the rapid Aptamer Selection Express (ASExp) that combines the interaction of a target with double-stranded (ds)-DNA and magnetic separation technology or non-equilibrium capillary electrophoresis of equilibrium mixtures (NECEEM) which, unlike SELEX, includes no amplification between repeated steps of selection. Aptamers may also be designed with specific affinity to cells, tissues and organisms. Binding of the nucleic acid aptamers to their specific target is governed by electrostatic, hydrogen bonding hydrophobic interactions, and also through Watson/Crick base pairing or triple helix binding, in a case where the target molecule comprises a polynucleotide.

Aptamers offer many advantages as molecular recognition molecules and are thus potentially attractive components of a plethora of analytical applications, such as the food quality testing, environmental safety testing and in vitro diagnostics. The advantages offered by aptamers include, for example, the fact that the design and development of aptamers does not involve animals or animal cells, thus expanding their applications to molecules that are not well tolerated physiologically. Another advantage concerns the fact that, since selection of aptamers is carried out in a test tube, the selection conditions of aptamers can be manipulated to yield aptamers that bind their targets under pre-specified conditions (such as of salt, temperature and pH). Furthermore, aptamers can be selected against a very wide variety of targets, from proteins to small molecules, independently of the in vivo immunogenicity of these molecules and in a shorter time period than that required for in vivo selection.

An additional advantage lies in the fact that aptamers often have high specificity and are able to discriminate between closely related targets (e.g. proteins from the same gene family) on the basis of subtle structural differences. Still further, aptamers are produced by cheaper chemical synthesis processes that are accurate and reproducible and generate materials with little or no batch to batch variation. Chemical synthesis can be readily scaled as required to meet production demand (e.g. for preparative uses). Whereas difficulties in scaling production limit the availability of some biologics, the cost of goods for aptamer synthesis at the kilogram scale is comparable to that for a single gram of antibodies. This gives the use of aptamers an advantage in preparative applications that require large quantities of targeting molecules, for example large scale affinity chromatography. Another advantage is that aptamers (especially DNA aptamers) are stable, are intrinsically adapted to regain activity following exposure to heat, denaturants, pH variations, etc. which enables both their re-use and their storage for extended periods of time (for example, over one year) at room temperature, in contrast to antibodies which cannot be regenerated and must be refrigerated. This advantage is particularly significant when contemplating reagents for large scale purification/decontamination and diagnostic kits that are intended for use at the point of contact, rather than in a laboratory setting where reagents can be stored under controlled conditions. Still further, aptamers are smaller than antibodies: whereas a typical aptamer may be 10-20 kDa in molecular weight (MW), equivalent to 30-60 nucleotides, antibodies have a MW of approximately 150 kDa. Their small size and batch to batch consistency potentially make them useful in designing multiplex assays which would not need to be re-calibrated for each batch. In addition, unlike antibodies, which often elicit an immune response when used in vivo, aptamers as a class have demonstrated little or no toxicity or immunogenicity.

The increased sensitivity and efficiency offered by the methods of the invention is achieved by combining aptamers that are the target recognition elements, with dendrimers that serve in signal amplification.

As a class, dendrimers are complex, highly branched molecules built from interconnected natural or synthetic monomeric subunits. A dendrimer used by the methods and compositions of the invention is constructed from DNA monomers, each of which is made from two DNA strands that share a region of sequence complementarity located in the central portion of each strand. Monomers are combined during the manufacturing process to prepare DNA dendrimers of different sizes and shapes. In order to prevent DNA dendrimers from falling apart over time, chemical “spot welds” are added to the growing assembly during the process using UV light via the intercalation and activation of psoralen cross-linkers. Dendrimers are purified according to their size and molecular weight on denaturing sucrose gradients after ultracentrifugation.

DNA dendrimers have the ability to be covalently and non-covalently bound to a large variety of different types of molecules and particles, for example, detectable or trapping moieties.

The present invention provides efficient and specific purification methods for removing or reducing the level of one or more contaminants, analytes or other targets present in a substance or any preparation or sample thereof, at greater efficiency than would be obtained by use of contaminant- or analyte-specific aptamers alone. In this application, the multiple labels attached to the aptamer via its conjugated dendrimer, enables effective trapping, immobilization, partitioning and removal of the analyte (target-compound)/aptamer-dendrimer complex from the body of the sample.

Further applications of the invention include sensitive detection assays for the detection and quantification of a target compound in a substance or any preparation or sample thereof. One detection protocol encompassed by the invention may be the two-site binding assay, or sandwich assay. Sandwich assays can be in a plate or also in lateral flow format. In the latter case, the target may first be bound by the aptamer/dendrimer conjugate and then by an immobilized capture element. As indicated herein above, the invention further provides sandwich assays based on the use of the aptamer-dendrimer conjugates of the invention, optionally, together with a capture composition, as described herein after for some embodiments. These nucleic acid aptamer-dendrimer based sandwich assays, designed with two or more sets of capture and target-recognition molecules and with corresponding labeled dendrimer detection molecules allow simultaneous multiplexed analysis of a mixture of target proteins in a single sample. Further applications of the invention include detection of target analytes by lateral flow, flow through and other point of use technologies whereby the addition of labeled dendrimers increases the sensitivity of detection through multiplication of the signaling molecule and/or through complexing of different anti-analyte aptamers. Another application of the invention includes a sensitized method to enable in vitro and in vivo imaging and tracking of aptamer targets by use of an aptamer with a bound dendrimer for recognition and detection.

In accordance with the invention, one or more target-specific aptamer molecules and a highly labeled polymeric molecule, specifically, a dendrimer, that is bound to one or more of the same or different aptamer molecules are provided for use in any desired applications including, but not limited to, applications of food or environmental, medical or molecular biological interest such as detection and quantification assays, diagnostic tests, reagent kits, components of visualization and imaging assays, reagents for trapping, removal or reduction of contaminants and so forth.

Thus, in the first aspect, the present invention provides a method for trapping, removing, detecting or quantification of at least one target compound in or from a substance or any preparation or sample thereof. The method of the invention comprises several steps:

The first step (a) involves providing at least one target-recognition composition comprising at least one nucleic acid-based aptamer that specifically recognizes and binds the at least one target compound, or complex thereof, or any parts or fragments thereof, or any combinations thereof, and at least one nucleic acid based dendrimer. In certain embodiments, specifically where trapping, removal, quantitation or detection of the target compound is desired, the dendrimers may be labeled with at least one detectable and/or trapping and/or partitioning moiety.

In the next step (b), the substance, or any preparation, sample, or an aliquot thereof is contacted with at least one of the target-recognition composition of (a) under conditions suitable for recognition and binding of the at least one target compound or any parts or fragments thereof, or any combinations thereof, by the at least one nucleic acid based aptamer to form at least one target-aptamer complex. The last step (c) comprises binding, trapping or detecting the at least one trapping or detectable moiety in the labeled dendrimer comprised within the target-recognition composition, which dendrimer is bound to or conjugated with the at least one aptamer in the target-aptamer complex. Such binding or alternatively, detection of the labeling moiety enables trapping, partitioning, removing from, or determination of the presence and optionally, the amount of said target compound in said substance.

As indicated above, an essential step of the method of the invention involves contacting of the target recognition composition of the invention with the tested substance or sample. The term “contacting” means to bring, put, incubate or mix together. As such, a first item is contacted with a second item when the two items are brought or put together, e.g., by touching them to each other or combining them. In the context of the present invention, the term “contacting” includes all measures or steps which allow interaction between the at least one of the target recognition composition and at least one target compound in the tested sample. The contacting is performed in a manner so that the at least one of the aptamers in the target recognition composition or alternatively, at least one capture composition can interact with or bind to the target compound in the tested substance or sample thereof. The binding may be non-covalent, reversible binding, e.g., binding via salt bridges, hydrogen bonds, hydrophobic interactions or a combination thereof.

To bring the sample in contact with the target recognition composition, the sample is mixed with the target recognition composition or at least with the aptamers comprised therein and incubated for a period of time sufficient for the binding to occur. Experts are usually familiar with the duration of the incubation step. This step can be optimized by determining the quantity of immobilized/trapped/detected target compound on the solid support at different points in time. Incubation times of between 10 seconds and up to about 3 hours or more can be appropriate for different target compounds.

According to one particular embodiment, the invention provides a method for trapping, partitioning, or removing at least one target compound in or from a substance or any preparation or sample thereof. The method of the invention comprises several steps:

The first step (a) involves providing at least one target-recognition composition comprising at least one nucleic acid-based aptamer that specifically recognizes and binds the at least one target compound, or complex thereof, or any parts or fragments thereof, or any combinations thereof, and at least one nucleic acid based dendrimer. In certain embodiments, the dendrimers may be labeled with at least one trapping and/or partitioning moiety.

In the next step (b), the substance, or any preparation, sample, or an aliquot thereof is contacted with at least one of the target-recognition composition of (a) under conditions suitable for recognition and binding of the at least one target compound or any parts or fragments thereof, or any combinations thereof, by the at least one nucleic acid based aptamer to form at least one target-aptamer complex. The last step (c) comprises binding and trapping the at least one trapping moiety in the labeled dendrimer comprised within the target-recognition composition, which dendrimer is bound to or conjugated with the at least one aptamer in the target-aptamer complex. Such binding of the labeling moiety enables trapping, partitioning or removing of said target compound from the substance.

The invention thus describes a partitioning method based on the use of aptamers bound to dendrimers for immobilization and removal or reduction of a molecular target upon exposure of a substance containing that target to the aptamer-dendrimer complex of the invention, providing a purified target substance, as well a substance from which the target has been removed or reduced in quantity.

The present invention describes methods for performing novel trapping and detection assays employing a nucleic acid reporter ligand and dendrimer detection molecule. As one example, the present invention can be applied in a direct assay, for example, a simple (direct, one-site) ELISA-type assay, as demonstrated in Examples 1-4, or alternatively, in a novel sandwich-type assay. Examples 5 and 6 demonstrate the feasibility of trapping a target compound using an aptamer-dendrimer composition labeled with biotin that is used herein as the trapping or partitioning moiety. As shown by these examples, targets were trapped using a NeutrAvidin Agarose Resin as a solid support and thereby removed from the substance.

It should be further appreciated that the method of the invention may be further used for inactivation of a hazardous molecular target contained in a substance for example, by partitioning the target compound-target recognition composition into inactive complexes and/or blocking one or more active sites of the hazardous target.

In yet another particular embodiment, the invention provides a method for the detection or the quantification of at least one target compound in a substance or any preparation or sample thereof. The method of the invention comprises several steps: The first step (a) involves providing at least one target-recognition composition comprising at least one nucleic acid-based aptamer that specifically recognizes and binds the at least one target compound, or complex thereof, or any parts or fragments thereof, or any combinations thereof, and at least one nucleic acid based dendrimer. In certain embodiments, the dendrimers may be labeled with at least one detectable moiety.

In the next step (b), the substance, or any preparation, sample, or an aliquot thereof is contacted with at least one of the target-recognition composition of (a) under conditions suitable for recognition and binding of the at least one target compound or any parts or fragments thereof, or any combinations thereof, by the at least one nucleic acid based aptamer to form at least one target compound-target recognition composition complex. The last step (c) comprises detecting the at least one detectable moiety in the labeled dendrimer comprised within the target-recognition composition. The dendrimer is bound to or conjugated with the at least one aptamer in the target-aptamer complex. Such detection of the labeling moiety enables determination of the presence and optionally, the amount of said target compound in said substance.

Still further, herein in certain embodiments, the inventors demonstrate a one-site (direct) ELISA-like assay for detection of a target compound, in which the target has been bound to a microtitre plate at different amounts by incubating solutions with different amounts on the plate and detection was carried out by biotinylated aptamers, or aptamer-dendrimer conjugates with multiple biotins attached to the dendrimers. The inventors show clearly that even without prior calibration and optimization of the experimental system, the aptamer-dendrimer conjugate employed in the direct ELISA-like assay according to the invention provides superior sensitivity as compared to a direct ELBA assay employing aptamers alone instead of aptamer-dendrimer conjugates.

As indicated above, in certain embodiments the method of the invention may be also applied in a form of two-site binding assays. The most widely used sandwich assays are based on the binding of two molecules, a reporter molecule and a capture molecule, to an analyte or target. In a sandwich assay, typically, the capture molecule is attached to a solid support. A substance that may contain the target compound is applied and allowed to react with the capture molecule. After washing, the reporter molecule is added to react with the target and the detection system indicates that an interaction has occurred. The target or antigen is, thus, “sandwiched” between the two layers of molecules, traditionally antibodies. This technique and all variations thereof, specifically, sandwich assays, optionally, in a lateral-flow format that may be applied either using a direct (non-competitive) or alternatively competitive procedures, are adaptable to the procedure described herein. The present invention therefore provides a sandwich-type detection assay based on the addition of a capture composition comprising an amino acid-based capture element, for example an antibody, or a nucleic-acid based capture element comprising, for example, an aptamer. This case results in a capture and recognition compositions which both comprise aptamers, and an aptamer-dendrimer conjugates in the target-recognition composition. The capture and the recognition compositions recognize two independent sites on a target molecule or optionally, one site on the target molecule and a second site on the target molecule:capture molecule complex or optionally one site on the target molecule and a second site on the target molecule:target recognition composition complex. The present invention also contemplates the detection of a specific target or targets using a sandwich assay of increased sensitivity in which the signal has been amplified through the use of multiply-labeled polymers which attach to target-specific aptamer molecules and act as label delivery molecules. The possibility of using dendrimers to add one or more orders of magnitude to the sensitivity of detection by aptamer based binding in the sandwich assay and in other assays further strengthens the future application of aptamers as a useful class of reagents for diagnostics. The aptamer in the present invention binds the target on the one hand, and other molecules which generate a signal (dendrimer) on the other. Therefore, the aptamers of the present invention are often termed “targeting molecules”.

Many detection assays, such as sandwich ELISA, flow through or lateral flow, and contaminant-removal or isolation procedures require the binding of the target to a solid support or a partitioning moiety, both of which allow the isolation of the target from other components of a given sample. To fulfill these requirements, according to some embodiments the method of the invention further comprises the steps of:

(i) providing at least one capture-composition comprising at least one nucleic acid based or amino-acid-based recognition element that specifically recognizes and binds at least one of the target compound or any parts or fragments thereof, or any combinations thereof. The at least one capture-composition is attached to a solid support or a partitioning element; and
(ii) contacting the substance or any preparations or samples thereof with at least one of the capture-composition of (i) under conditions suitable for recognition and binding of the at least one target compound or any parts or fragments thereof, or any combinations or complex thereof by the at least one nucleic acid based or amino-acid-based recognition element comprised within the capture-composition to form at least one complex comprising the target compound or any complex thereof and the capture-composition attached to said solid support or partitioning element.

As indicated herein before, it is understood that for contaminant removal or trapping, the invention may use a target recognition composition where the dendrimer is labeled with a trapping or partitioning moiety. For example, multiple ferritins or biotins that can be caught with a magnet or with avidin. In other alternative embodiments, the capture-composition provided by the invention that comprises a nucleic acid or amino acid based recognition element specifically recognizing and binding the target compound, may be also applicable for target removal allowing both detection and removal of the contaminant. It should be also appreciated that the use of an additional element such as the capture composition may enable partitioning the target molecule forming complexes that may inactivate the target compound.

According to some specific embodiments, the invention provides a method for detection or quantification of at least one target compound in a substance or any sample thereof, which comprises the steps of:

The first step (a) requires providing at least one target-recognition composition comprising at least one nucleic acid based aptamer that specifically recognizes and binds the at least one target compound, or any parts or fragments thereof, or any combinations or complexes thereof. Specific examples may include any complexes thereof with the capture-composition, or in case of a competition assay, any combination or complex thereof with a further tagging element, e.g. with BSA. The target recognition composition further comprises at least one nucleic acid based dendrimer labeled with at least one moiety, specifically, a detectable moiety.

The next step (b) requires providing at least one capture-composition comprising at least one nucleic acid based or amino-acid-based recognition element that specifically recognizes and binds at least one of the target compound or any parts or fragments thereof, or any combinations thereof. It is noted that the at least one capture-composition may be attached to a solid support or a partitioning element.

The next step (c) involves contacting the substance or any preparation or sample thereof with at least one of the capture-composition of (b) under conditions suitable for recognition and binding of the at least one target compound or any parts or fragments thereof, or any combinations thereof, by the at least one nucleic acid based or amino-acid-based recognition element comprised within the capture-composition to form at least one target compound-capture-composition complex attached to the solid support or partitioning element.

In the following step (d) the at least one target compound-capture-composition complex obtained in step (c) is contacted with at least one of the target-recognition composition of (a) under conditions suitable for recognition and binding of the at least one target compound, or complex thereof, or any parts or fragments thereof, or any combinations thereof in the target compound-capture-composition complex by the at least one nucleic acid-based aptamer comprised within the target-recognition composition. This forms at least one target recognition composition-target compound—capture composition complex attached to the solid support or partitioning element.

The final step (e) comprises detecting the at least one detectable moiety in the labeled dendrimer comprised within the target-recognition composition, that is bound to or conjugated with the at least one aptamer in the target recognition composition-target compound-capture composition complex attached to the solid support or partitioning element. It should be noted that the detection and the determination of the amount of the at least one detectable moiety, indicates the presence and optionally, the quantity of the target compound in the examined substance or any preparation or sample thereof.

Thus, in this specific embodiment, the present invention provides a method, optionally in a lateral follow format, for detecting the presence of a target compound in a substance which may contain said target compound comprising: (a) exposing or contacting a substance which may contain said target compound to a capture molecule capable of binding to said target molecule, wherein the capture molecule comprised in the capture composition is immobilized on a solid support; (b) optionally, removing the remainder of the substance from said capture molecule:target molecule complex; (c) adding to said capture molecule-target molecule complex a targeting molecule (e.g., the aptamer comprised within the target-recognition composition of the invention) capable of binding to said target molecule; and (d) detecting said capture molecule:target molecule:targeting molecule (specifically, aptamer) complex. In specific embodiments, the capture molecule may be an antibody or aptamer and targeting molecule is a nucleic acid ligand, specifically, aptamer specific for said target molecule. The targeting molecule is attached to a dendrimer labeled with at least one detectable moiety. It should be appreciated that such detection may provide both quantitative and qualitative information. Moreover, such assay may be performed in both, the direct manner indicated above, or alternatively, in a competitive manner.

In a specific example of such competition assay, a capture element specific for a certain target compound, for example, a pesticide is attached to a solid support (e.g. nitrocellulose, beads, etc.). Samples with an unknown amount of the target pesticide are applied and allowed to mix with a known amount of the same target labeled with a labeling element (e.g. BSA, biotin etc.). An aptamer/dendrimer conjugate that specifically recognizes the labeling element, is applied, and allowed to contact the sample. The aptamer/dendrimer conjugate, for example, an anti-BSA aptamer/dendrimer conjugate is labeled with a detectable moiety, e.g. gold beads to form a gold beads-dendrimer-anti-BSA aptamer-BSA-pesticide complex. The tested substance is allowed to move over the capture element which captures both the target compound (pesticide) and the labeled-target (pesticide-BSA complex). The lower the signal from the dendrimers with the anti-BSA aptamers, the more pesticide there was in the sample.

In particular embodiments, the capture molecule must bind to the target molecule to form a capture molecule:target molecule complex. The targeting molecule, specifically, aptamer comprised within the target-recognition composition of the invention, must also bind to the target molecule or to the capture molecule:target molecule complex, and additionally must bind or be conjugated to a dendrimer-based detection system, forming said target-recognition composition, wherein a capture molecule:target molecule:targeting molecule complex can be identified and optionally, quantified.

Consideration is also given to alternative embodiments using for example, a lateral flow format, where the sample containing the target molecule is first contacted with a targeting molecule (aptamer) bound or conjugated to a dendrimer-based detection system (that is the target-recognition composition of the invention), forming a target recognition composition:target compound complex, and then contacted with a capture molecule, forming a capture composition:target compound:target recognition composition complex identifiable by said dendrimer-based detection system. More specifically, according to some specific embodiments, the invention provides an alternative method for detecting or quantitating at least one target compound in a substance or any sample thereof, which comprises the steps of:

The first step (a) requires providing at least one target-recognition composition comprising at least one nucleic acid based aptamer that specifically recognizes and binds the at least one target compound, or any parts or fragments thereof, or any combinations or complexes thereof, and at least one nucleic acid based dendrimer labeled with at least one moiety; specifically, a detectable moiety.

The next step (b) requires providing at least one capture-composition comprising at least one nucleic acid based or amino-acid-based recognition element that specifically recognizes and binds at least one of the target compound or any parts or fragments thereof, or any combinations thereof. It is noted that the at least one capture-composition may be attached to a solid support or a partitioning element.

The next step (c) involves contacting the substance or any preparation or sample thereof with at least one of the target-recognition composition of (a) under conditions suitable for recognition and binding of said at least one target compound or any parts or fragments thereof, or any combinations thereof, by the at least one nucleic acid based aptamer in the target recognition composition to form at least one complex of target compound with said target-recognition composition.

The next step (d) involves contacting the at least one target compound-target-recognition composition complex obtained in step (c) with at least one of the capture-composition of (b) under conditions suitable for recognition and binding of said at least one target compound, or complex or combination thereof, in said target compound-target recognition-composition complex by said at least one nucleic acid based or amino-acid-based recognition element comprised within said capture-composition. Such contact results in formation of at least one target recognition composition-target compound-capture-composition complex attached to said solid support or partitioning element.

The final step (e) involves detecting the at least one detectable moiety in the labeled dendrimer comprised within said target-recognition composition, that is bound to or conjugated with said at least one aptamer in said target recognition composition-target compound-capture-composition complex attached to said solid support or partitioning element. It should be noted that the detection and/or determination of the amount of the at least one detectable moiety indicates the presence and/or quantity of said target compound in the examined substance or any preparation or sample thereof.

It should be further appreciated that such version of lateral flow assay may also be performed by both, a direct assay or alternatively, an indirect competition assay. In a competition assay, a sample containing an unknown amount of a specific target compound is allowed to mix with a known amount of a target compound labeled with a specific labeling element, for example, BSA. The mixture of both is then contacted with the target-recognition composition that comprises an aptamer that specifically recognizes and binds the target compound. The mixture is then contacted with a capture composition that may be an antibody or an aptamer, attached to a solid support. It should be noted that in this embodiment, the capture composition is specific for the labeling element (BSA for example) and therefore, only target recognition compositions that are attached to a labeled target (BSA-target) will bind the capture molecule. Thus, the lower the signal from the captured complexes, the more target compound was in the sample.

It should be appreciated that the target-recognition composition of the invention provides a variety of different applications. Thus, the invention also encompasses a visualization method based on the use of aptamers bound to dendrimers for imaging of targets in vitro or in vivo, a partitioning method based on the use of aptamers bound to dendrimers for immobilization and removal or reduction of a molecular target upon exposure of a substance containing that target to the aptamer-dendrimer complex and an inactivation method based on the introduction of inactivating aptamers bound to dendrimers to a substance containing a hazardous molecular target.

Thus, in some specific embodiments, the method of the invention may be particularly suitable in cleaning, decontamination and purification applications. More specifically, the methods of the invention provide trapping, partitioning for the removal or alternatively, for purification and enrichment of at least one target compound in/from a substance or any sample or preparation thereof. In further specific embodiments, the method may be used for removing at least one target compound from a substance or any sample thereof. The method of the present invention is therefore applicable for the removal or reduction of one or multiple target compounds or analytes from foods, water, environmental discharges such as liquid wastes, solutions in which controlled chemical reactions are being carried out or other substances. The removal or reduction is carried out by exposure of the substance which may contain the target compound to an aptamer molecule or molecules which recognize that target and is/are attached to a dendrimer molecule which will enable immobilization of the target/aptamer-dendrimer complex on a solid substrate, specifically, via its partitioning element. In some cases, the invention further provides exposure of food, water or environmental discharges to a number of different aptamers which are attached to the same or different dendrimers.

As indicated above, in certain embodiments the invention relates to specific trapping of a target compound contained in a substance. The term trapping includes catching, capturing, binding and thereby immobilizing the captured or trapped molecule attaching it to a solid support or a partitioning element.

The term “partitioning”, with respect to a target compound, refers to the separation of said target from the remainder of the substance or any sample thereof to produce a substance devoid of said target compound. The term “partitioning” encompasses therefore depletion and removal of the target compound from the substance. The term “depleting” or “depletion” as used herein is defined as the removal of the target compound from the substance used to such extent so as to obtain a decontaminated substance that can be determined by a person of ordinary skill in the art. More specifically, the term “removal” or “depletion”, as used herein, either by partitioning or trapping of the target compound, means the restriction, reduction, decrease or diminishing of the amount of the target compound in the substrate or any sample or preparation thereof by at least about 1%400%, about 5%-95%, about 10%-90%, about 15%-85%, about 20%-80%, about 25%-75%, about 30%-70%, about 35%-65%, about 40%-60% or about 45%-55%. Said restriction, retardation, reduction, decrease or diminishing of the amount of the target compound in a substance also be by at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or about 100%. As shown by Example 5, trapping of the target compound (thrombin) by the target recognition composition of the invention resulted in reduction of the amount of thrombin to about 40% of the original amount (200 ng final, where the initial amount was 500 ng), thereby leading to a substance that is about 60% free, purified or depleted of or from the contaminating target compound. It should be noted that repetitive purification and removal cycles are also contemplated by the invention. As used herein, the term “purified” or “to purify” refers to the removal of the target compounds that may be any contaminants from a substance or any sample thereof.

In yet certain alternative embodiments, it must be understood that the trapping procedure provided by the invention may be used for isolation, purification and enrichment of a desired target compound present in a substance. More specifically, partitioning of the target compound from the examined substance may be used to purify the target from any other compounds of the substance. In such alternative embodiment the term “partitioning” may also encompass enriching of the target compound. It should be understood that purification or enrichment mean increasing, enhancing or elevating the relative amount of a specific target compound in a substance by trapping said particular target compound while other compounds are removed from the substance.

It should be further noted that for partitioning purpose, the invention may use only the target recognition composition were the partitioning moiety is bound or connected to the dendrimers. In certain embodiments, this may be applied in a flow-through format

However, the use of an additional element such as capture composition attached to a solid support or partitioning element may also be applicable, providing both trapping and identification of the target compound.

In other embodiments, the method of the invention may be specifically applicable for detecting or quantification of at least one target compound in a substance or any sample thereof. Thus, the method of the present invention may be suited for the detection of target compounds or analytes in foods, water and environmental discharges such as liquid wastes, in addition to clinical diagnosis of physiologic conditions in both human and veterinary diagnostics. In the case of in vitro diagnostics, the substance is usually a biological material that may or may not contain the target compound of interest. Such biological materials include blood, plasma, serum, sputum, urine, semen, cerebrospinal fluid, bronchial aspirate, and macerated tissue. It should be noted that the method of the invention may be used for detection and quantification applying the target recognition composition alone or the target composition having dendrimer/s labeled with a detectable moiety together with a capture composition attached to a solid support. It must be recognized that both options may be applied either in a direct or a competition manner and may provide quantitative and/or qualitative information.

As indicated herein above, the method provided by the invention is particularly suitable for determining and assessing the amount of a target compound present in a substance. Reference to “determining”, as used herein, includes estimating, quantifying, calculating or otherwise deriving by measuring an end point indication that may be for example, the appearance of a detectable moiety, any detectable change in the levels or any change in the rate of the appearance or alternatively, the disappearance of the detectable moiety.

As used herein, “assessing” refers to quantitative and/or qualitative determination of the detectable moiety attached to the dendrimer used by the invention, e.g., obtaining an absolute value for the amount or concentration of the target compound, and also of obtaining an index, ratio, percentage, visual or other value indicative of the level of the target compound in the examined substance or sample thereof.

In some envisioned embodiments of the invention, the method is for the visualization of target compounds in vitro, on membranes, in cell culture, tissue sections, whole mounts or in vivo by the exposure of cells, tissues or organs that may contain the target to an aptamer reporter molecule which recognizes that target and is attached to a dendrimer detection molecule and then carrying out visualization of the aptamer-dendrimer complex. Importantly, the discussed dendrimers are stable in-vivo [WO/2010/017,544 Kadushin J M and Getts R C, 2009] and therefore suited for in vivo detection and visualization of targets.

Yet further embodiments consider the method of the invention for specifically inactivating at least one target compound in a substance or any sample or preparation thereof. This may be achieved when a sample is connected with the target-recognition composition of the invention that comprises dendrimers attached to different aptamers specific for the target compound. Binding of a target to the different aptamers that are arranged on a dendrimers in a specific spatial configuration, may completely block active sites or force structural changes on the target compound leading to its inactivation. In still further specific embodiments, partitioning and trapping of the target compound as provided in some embodiments of the invention may lead to creation of complexes of the target compound thereby leading to inactivation thereof. In still further alternative embodiments, change in the spatial configuration of a target molecule may lead to its activation. Therefore, the method of the invention may be suited for activating at least one target compound in a substance or tissue or any sample thereof.

As noted above, specifically for detection and quantification purpose, the method of the invention further involves the use of a capture composition allowing both lateral flow and flow through assay formats. In various embodiments of the method of the invention, the nucleic acid based or amino-acid-based recognition element comprised within the capture-composition may be any one of a nucleic acid selected from: aptamer, probe, primer, or oligonucleotide specific for the target, or an amino-acid selected from: an antibody, peptide, ligand, receptor or substrate, or any small molecule specific for the target.

According to one specific embodiment, the capture composition used by the invention may comprise a nucleic acid based recognition element, specifically, an aptamer.

In yet another embodiment, the capture composition used by the invention may comprise an amino acid based recognition element, specifically, an antibody.

It should be further noted that in cases that competition assay is applied, specifically, a sandwich assay involving both the capture composition and the target recognition composition, the target recognition elements in both compositions may recognize different molecules. For example, the capture composition may specifically recognize the target compound, where the target-recognition composition recognizes a labeling element, for example, BSA that may be attached to a known and predetermined amount of the same target compound.

As to the target recognition composition that comprises aptamers and dendrimers, it must be acknowledge that the combination of both elements provides a large variety of optional structures. First, it is appreciated that the aptamer and dendrimer comprised in the target-recognition composition may be attached or conjugated prior to contacting the composition with the substance or any sample thereof, after or simultaneously with, contacting the composition with the substance or any sample thereof. Thus, according to certain specific embodiments, the aptamers comprised within the target-recognition composition of the invention are attached or conjugated to the dendrimers before the composition is contacted with the substance or sample.

Still yet further embodiments encompass target recognition compositions having dendrimers that are attached or connected with the aptamers only after the composition is contacted with the sample. This is made possible due to the chemical nature of the two components, i.e., nucleotide complementarity between the aptamer and dendrimer may guide them into correct interaction and generate correct attachment despite the presence of many other components in a given sample or solution.

In certain embodiments, the method of the invention is contemplated, wherein each dendrimer specifically recognizes and binds a specific aptamer or a specific aptamer-target complex.

As used herein, the term “aptamer” refers to a nucleic acid molecule that may selectively interact with a non-oligonucleotide or oligonucleotide molecule or group of molecules. In various embodiments, aptamers may include single-stranded, partially single-stranded, partially double-stranded or double-stranded nucleic acid sequences; sequences comprising nucleotides, ribonucleotides, deoxyribonucleotides, nucleotide analogs, modified nucleotides and nucleotides comprising backbone modifications, branch points and non-nucleotide residues, groups or bridges; synthetic RNA, DNA and chimeric nucleotides, hybrids, duplexes, heteroduplexes; and any ribonucleotide, deoxyribonucleotide or chimeric counterpart thereof and/or corresponding complementary sequence. In certain specific embodiments, aptamers used by the invention are composed of deoxyribonucleotides. As used herein, aptamers may also be referred to as nucleic acid ligands.

It is understood that the aptamers of the invention may comprise between about 5 to about 100 nucleotides. More specifically, aptamers may comprise between about 7-98 nucleotides, or about 9-96 nucleotides, or about 11-94 nucleotides, or about 13-92 nucleotides, or about 15-90 nucleotides, or about 17-88 nucleotides, or about 19-86 nucleotides, or about 21-84 nucleotides, or about 23-82 nucleotides, or about 25-80 nucleotides, or about 27-78 nucleotides, or about 29-76 nucleotides, or about 31-74 nucleotides, or about 33-72 nucleotides, or about 35-70 nucleotides, or about 37-68 nucleotides, or about 39-66 nucleotides, or about 41-64 nucleotides, or about 43-62 nucleotides, or about 45-60 nucleotides, or about 47-58 nucleotides, or about 12-48 nucleotides, or about 14-46 nucleotides, or about 15-44 nucleotides, or about 16-42 nucleotides, or about 18-40 nucleotides, or about 20-38 nucleotides, or about 22-36 nucleotides, or about 24-34 nucleotides, or about 26-30 nucleotides. In particular embodiments, the aptamers used by the invention may comprise about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 711, 72, 73, 74, 75, 76, 77, 78, 79, 80, 81, 82, 83, 84, 85, 86, 87, 88, 89, 90, 91, 92, 93, 94, 95, 96, 97, 98, 99 or -100 nucleotides. In more specific embodiments, the aptamers may comprise 15 nucleotides. In yet another example, the aptamer may comprise 27 nucleotides. It should be noted that the nucleotides are deoxynucleotides.

As noted above, in certain embodiments the aptamers and dendrimers used by the method of the invention comprise nucleic acids. The term “nucleic acid” is referred to often herein, and relates to DNA, RNA, single-stranded or double-stranded, and any chemical modifications thereof. Modifications include, but are not limited to, those which provide other chemical groups that incorporate additional charge, polarizability, hydrogen bonding, electrostatic interaction, and functionality to the nucleic acid ligand bases or to the nucleic acid ligand as a whole. Such modifications include, but are not limited to, 2′-position sugar modifications, 5-position pyrimidine modifications, 8-position purine modifications, modifications at exocyclic amines, substitution of 4-thiouridine, substitution of 5-bromo or 5-iodo-uracil; backbone modifications, methylations, unusual base-pairing combinations such as the isobases isocytidine and isoguanidine and the like. Modifications can also include 3′ and 5′ modifications such as capping.

As indicated above, in specific embodiments, the dendrimers used by the invention are highly labeled polymeric nucleic acid molecules as described in patents of Genisphere [U.S. Pat. Nos. 5,175,270; 5,484,904; 5,487,973] which display great versatility in their use in conjunction with oligonucleotide aptamers as described herein. The polymeric molecule is constructed from at least three types of single stranded DNA or RNA molecules attached together to form a polymeric scaffold. The polymer carries at least one label molecule, which generates a detectable signal. Within each polymer, at least one type of single stranded nucleic acid molecule is provided which serves as a labeling site or a label-binding site. For example, the labeling molecules may be suitably designed DNA or RNA oligonucleotides capable of binding to the polymeric scaffold. Label-binding nucleic acid molecules includes molecules designed to bind or carry one or more suitable label moieties, and also includes molecules capable of binding to other molecules that themselves incorporate or bind labels. Furthermore, psoralen intercalation between the double stranded regions of the DNA dendrimer, followed by UV irradiation, provide for fully covalent bonds between the hybridized regions of the DNA dendrimers. In certain specific embodiments, the dendrimers used by the invention are composed of deoxyribonucleotides.

The DNA dendrimers encompassed by the invention include nonspherical and three-dimensional as well as spherical DNA dendrimers. The three-dimensional or spherical DNA dendrimers used by the invention may be one-layer, two-layer, three-layer, four layers, five-layer, six-layer, seven-layer, eight-layer, nine-layer or even ten-layer or more DNA dendrimers. In a first specific embodiment, the dendrimers of the invention may comprise at least two-layers, at least three-layers or at least four-layers. In another specific embodiment, the dendrimers used by the invention comprise at least two layers, specifically, such dendrimers are two-layer dendrimer. In yet another specific embodiment, the dendrimers used by the invention may be four-layer dendrimers. It should be noted that non-spherical and linear DNA dendrimers provide a more compact structure then three-dimentional dendrimers. It should be further noted that the number of layers in a three-dimentional DNA dendrimers or the number of strands in a linear DNA dendrimers determines the size of the DNA dendrimers.

To accomplish the objectives of the invention, an effective means for attaching aptamers to dendrimers is necessary. Moreover, the inventors seek a controlled attachment thereof, where specific dendrimers will bind specific aptamers. In an example of such a system, one or more types of oligonucleotides may be provided in the polymer or dendrimer to facilitate its attachment to the aptamer which then acts as a delivery device for the polymer or dendrimer. Such targeting oligonucleotides are oligonucleotides of various lengths that have, at their terminus, a sequence provided for complementarity based recognition of and binding to the aptamer or an extension of the aptamer, either directly or via a bridging oligonucleotide molecule. The complementary sequence on the aptamer for recognition of the targeting oligonucleotide on the polymer or on a bridging molecule can be incorporated during synthesis of the aptamer. Hybridization of the complementary sequence of the aptamer to the complementary terminating oligonucleotide of the labeled polymer may be followed by cross-linking, resulting in labeling of the aptamer. Alternatively, attachment of the aptamer to labeled dendrimer may be carried out by ligation mediated by bridging molecules that recognize sequences that have been added to the aptamer and sequences that have been added to the dendrimer. Lastly, the nucleic acid ligand, specifically, aptamer, can be directly incorporated into the labeled polymer, specifically, dendrimer, by synthesis as part of a polymer monomer or terminating oligonucleotide.

In certain embodiments, the aptamer-dendrimer conjugates comprised within the target recognition composition of the invention may use a connecting spacer or tether. The term “spacer” or “tether” as referred to herein, relates to an oligonucleotide comprising between about 2-5 nucleotides, or about 5-10 nucleotides, or about 10-15 nucleotides, or about 15-30 nucleotides, or about 30-50 nucleotides, or about 50-70 nucleotides, or about 70-90 nucleotides, or about 90-150 nucleotides, or about 150-300 nucleotides. In particular embodiments, the spacer comprises about 10-50 nucleotides. In preferred embodiments, the nucleotides are deoxynucleotides.

Thus, it can be understood that the dendrimers may be either directly attached to the aptamers, for example where the aptamer sequence is simply incorporated into one of the dendrimer units or alternatively, attached to the aptamers via nucleic acid complementarity. The attachment of the dendrimers to the aptamers via nucleic acid complementarity may be followed by cross linking which brings about formation of a very stable complex. Other embodiments consider the attachment of the dendrimers to the aptamers via a bridging complementary nucleic acid followed by ligation, or by cross linking, such as UV cross linking.

The term “cross-linking” and “UV cross linking” as referred to herein relate to the establishment of chemical bonds that link one polymer chain, such as a nucleic acid, to another that can be carried out without or with prior psoralen intercalation, as described above. For example, the cross-linking may occur between dendrimers and aptamers, between dendrimers and bridging molecules, or between aptamers and bridging molecules. For UV cross-linking irradiation takes place using ultraviolet radiation in a wavelength range from 200 to 400 nm for a short time, using in particular high-pressure or medium-pressure mercury lamps with an output of from 80 to 240 W/cm. The intensity of irradiation is tailored to the particular quantum yield of the UV photoinitiator, to the degree of cross-linking which is to be established, and to establish the degree of orientation. A photoinitiator is any chemical compound that decomposes into free radicals when exposed to light. Protocols for UV cross-linking are well known in the art.

According to other embodiments, the dendrimers may also be attached to the aptamers via a biological affinity system. The term “biological affinity system” relates to known protein interactive partners which bind each other with high affinity and specificity and may be used as labels which facilitate the binding of molecules to which they are bound. A well known example of such a biological affinity system is the biotin-streptavidin or avidin system.

Non-limiting examples of other biological affinity systems include biotin-streptavidin, avidin or neutravidin and GST (Glutathione-S-transferase) and glutathione. The binding partners need not necessarily both be proteins. For example, such binding partners may be haemaglutinin tag and an anti-haemaglutinin antibody or antibody fragment, lectins and oligosaccharides, MBP and maltose, and others.

In various embodiments, the aptamer-dendrimer combinations according to the method of the invention may take different forms; the dendrimers may each be attached to or conjugated with more than one identical or different aptamers. According to more specific embodiments, the dendrimers may each be attached to or conjugated with more than one identical aptamers. It should be appreciated, that this specific embodiment applies to dendrimers used by any method described by the invention, including the sandwich assay method, that further use a capture-composition.

In particular embodiments, the invention relates to a method for detecting, trapping, quantitating, removing, inactivating, or activating at least one target compound in or from a substance or any sample thereof, wherein the dendrimers are each attached to more than one aptamer, more specifically, each dendrimer is attached to between about 2-60, specifically, 2-5 aptamers, or each dendrimer is attached to between about 6-9 aptamers, or each dendrimer is attached to between about 10-13 aptamers, or each dendrimer is attached to between about 14-17 aptamers, or each dendrimer is attached to between about 18-21 aptamers, or each dendrimer is attached to between about 22-25 aptamers, or each dendrimer is attached to between about 26-29 aptamers, or each dendrimer is attached to between about 30-33 aptamers, or each dendrimer is attached to between about 34-37 aptamers, or each dendrimer is attached to between about 38-41 aptamers, or each dendrimer is attached to between about 42-45 aptamers, or each dendrimer is attached to between about 46-49 aptamers, or each dendrimer is attached to between about 50-53 aptamers. Particularly useful for the method are dendrimers which are attached to between about 5-30 aptamers. In such a manner, multimeric and/or multivalent aptamer ligands are presented to a single or multiple target molecules.

In yet further specific embodiments, the dendrimers used for the target recognition composition of the invention may be each attached to between about 1 to about 50 aptamers, more specifically, about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 45, 46, 47, 48, 49, 40, 41 42, 43, 44, 45, 46, 47, 48, 49 or 50 aptamers. As demonstrated by the following Examples, the dendrimers of the invention may be each attached to between about 1, 5 or 20-30 aptamers. In yet further specific embodiments, the dendrimers are attached to one aptamer. Still further embodiments encompass the use of dendrimers each attached or connected to five aptamers. More specific embodiments relate to the use of dendrimers each attached to about 20-30 aptamers.

Furthermore, the aptamers bound to a dendrimer may be different aptamers recognizing different or identical targets.

In one embodiment, the aptamers bound to a dendrimer may be different aptamers that recognize identical targets. In this particular embodiment, several different aptamers attached to the same dendrimer are used to recognize the same target in an enhanced and more efficient manner. Such particular method may be applicable for use in detection, quantification, trapping, removing, inactivating or activating of at least one target compound in or from a substance or tissue or any preparation or sample thereof.

In an alternative embodiment, the aptamers bound to a dendrimer may be different aptamers that recognize different targets. Some aptamer-dendrimer configurations contemplated by the embodiments of the method of the invention are particularly advantageous for trapping, removing or inactivating of one or more targets in a sample. According to these embodiments, the target-recognition composition comprises at least one aptamer and at least one dendrimer. In certain embodiments, the aptamers are different aptamers recognizing different targets. This method allows a direct trapping, removing or inactivating of at least one target compound in or from a substance or tissue or any sample thereof.

In particular embodiments, the method of the invention may further comprise the step of contacting the sample with a capture-composition that recognizes a specific—aptamer-dendrimer-target complex, whereas in other embodiments the method of the invention may further comprise the step of contacting of the sample with a capture-composition that recognizes a specific target prior to the contacting of sample with target recognition composition. It should be appreciated that in case the different dendrimers are not labeled differently or in case that each dendrimer is attached to different aptamers that recognize different targets, such sandwich assay may further enable a rough “yes/no” detection. Such an assay may be used for example, in detection of different contaminants in a sample (different targets that are not necessarily labeled differently).

The invention provides further embodiments that are advantageous for multiplex assays, that is, the simultaneous detection and/or quantitation of more than one target compounds or analytes. This detection and/or quantitation may be performed in foods, water and environmental discharges such as liquid wastes, in addition to clinical diagnosis of physiologic conditions in both human and veterinary diagnostics. In some embodiments, the multiplex analysis may be accomplished by exposure of the substance to a surface upon which capture molecules for more than a single target have been immobilized. Following optional washing away of unbound substance, the capture molecule:target molecule complexes are exposed to aptamer targeting molecules recognizing each of the target molecules or the capture molecule:target molecule complexes. Detection is carried out through attachment of differently labeled dendrimers to each of the specific targeting aptamers.

According to such embodiments, the composition used by the method of the invention allows recognition of several different targets (multiplexing). Such composition may comprise at least two dendrimers, wherein each of the dendrimers is labeled with at least one identical detectable and/or trapping and/or partitioning moiety and wherein each of the dendrimers is attached or conjugated to at least one identical or different aptamer, the aptamers recognizing an identical target. In more specific embodiments, each different target is recognized by aptamers attached to differently-labeled dendrimers. Such assay therefore enables simultaneous detection and quantification of different targets.

It should be appreciated that in certain embodiments, multiplexing as used herein also encompasses the option where each multiplex signal (each “class” of dendrimer) represents a “family” of targets rather than a single target. Each group of targets (different targets belonging to a single group) may be recognized by dendrimers labeled with a unique label specific for this group.

Multiplexed analysis of a large number of analytes can also be performed on spatially addressable test sites. DNA chip arrays in which known DNA sequences are immobilized on a solid surface to capture and detect unknown sequences have been designed for genomic applications. Analogous to DNA arrays, aptamer and dendrimer conjugate arrays printed on chips or membranes or in microtitre plates or on other appropriate surfaces can be designed to analyze a number of targets where the geometric address of each detection signal will provide the target identity. This approach can be implemented to increase multiplexing ability for assays carried out in a microtitre plate and in the case of point of use lateral flow type diagnostic or detection systems in which single samples are analyzed simultaneously for more than one target and more than one label may be employed. According to one specific embodiment, such method may require a further step of adding a capture molecule attached to an array on a predetermined location.

As indicated above, for trapping, partitioning, detection and quantification, the dendrimer comprised within the target recognition composition used by the method of the invention is labeled with a detectable label or any trapping and/or partitioning moiety. The term “label moiety” or “label” as referred to herein, relates to a specific group of atoms within a molecule having the ability to either bind other known specific molecules including those which have associated with them a molecule that emits a detectable signal or emit a detectable signal itself. The label either emits or produces a detectable signal directly or through interaction with one or more additional members of a signal producing system. Any type of label can be used consistent with the invention, with conventional labeling methods known in the art being suitable.

The dendrimer molecule comprises a detection system which may include a wide array of known chemical entities. The detection system may be directly bound to the dendrimers or may be associated with the dendrimer following binding of the reporter molecules, with their conjugated dendrimers, to the target, as by a biotin-streptavidin association. The detection system can be an enzyme, a fluorophore, a radiolabel, or any other signal-emitting, signal inducing, or otherwise chemically or physically discernible. The various detection systems are well known to those skilled in the art. In one particular embodiment, the dendrimer molecule may be labeled with fluorescein or Alexa Fluor as reporter ligands. Alternatively, enzymes such as alkaline phosphatase or horseradish peroxidase (HRP) that could subsequently be used to generate fluorescence or chemiluminescence or a color reaction can also be conjugated to the dendrimer. Such enzyme conjugates are expected to further enhance the sensitivity of the assay. Enzymes or other appropriate reporter groups can be introduced to dendrimers either directly or indirectly, for example, through either biotin-streptavidin interaction or antigen-antibody interaction.

Examples for signal emitting molecules include, but are not limited to fluorescent labels (fluorophores), chemiluminescent labels, and ligands activating or inhibiting a detectable chemical reaction, enzymatic labels, and inorganic labels. The label is one that preferably does not provide a variable signal but instead provides a constant and reproducible signal over a given period of time.

The term “detectable” as used herein refers to the presence of a detectable signal generated from a detectable chemical reaction that is immediately detectable by observation, instrumentation, or film. The term “detectable moiety” as used herein refers to a moiety or tag causing an occurrence of, or a change in, a signal that is directly or indirectly detectable (observable) either by visual observation or by instrumentation. Typically, the detectable moiety is detectable in an optical property (“optically detectable”) as reflected by a change in the wavelength distribution patterns, or intensity of absorbance, or a combination of such parameters.

More specifically, labels that are directly detectable include, but are not limited to, labels detectable by fluorescent detection, chromogenic detection, and chemiluminescent detection, or radioactive labels. Non-limiting examples include fluorescent labels such as fluorescein, rhodamine, resorufin, and derivatives thereof, coumarins (such as hydroxycoumarin), BODIPY, cyanine dyes (e.g. from Amersham Pharmacia), Alexa dyes (e.g. from Molecular Probes, Inc.), fluorescent dye phosphoramidites, or so forth; and radioactive isotopes, such as ³²S, ³²P, ³H, etc. Or, marker enzymes such as alkaline phosphatase (AP), beta-galactosidase, or horseradish peroxidase can be used, which are detected using a chromogenic substrate. In the case of AP, for example, detection can be effected using 5-bromo-4-chloro-3-indolyl phosphate or a nitroblue tetrazolium salt. Inorganic labels can be used, such as colloidal gold particles or ferritin.

Examples of labels that provide a detectable signal through interaction with one or more additional members of a signal producing system include attachment moieties that specifically bind to complementary binding pair members, where the complementary binding pair members comprise a directly detectable label moiety, such as a fluorescent moiety as described above. For example, biotin can be used as a binding ligand with avidin or streptavidin as the receptor. One or more labels may be conjugated to the avidin or streptavidin, such as 5(6)-Carboxyfluorescein-N-hydroxysuccinimide ester (FLUOS), 7-amino-4-methyl-coumarin-3-acetic acid-N′-hydroxysuccinimide ester (AMCA, activated) and fluorescein isothiocyanate (FITC) which are available from Boehringer Mannheim of Indianapolis, Ind. Methods for fluorescently labeling proteins with fluorescent labels, and methods for detection of the fluorescent labels, are well known in the art. A variety of commercially available labeled streptavidin and avidin molecules are also available, including, for example, streptavidin-gold, streptavidin-fluorochrome, streptavidin-AMCA, streptavidin-fluorescein, streptavidin-phycoerythrin (STPE), streptavidin-sulforhodamine 101, avidin-FITC and avidin-Texas red. RTM which are commercially available from Boehringer Mannheim, Indianapolis, Ind. Another example of an attachment moiety is digoxygenin that can be recognized at high affinity with labeled anti-digoxygenin antibodies.

In certain specific embodiments of the invention, the labeled dendrimers of the invention may be labeled using a fluorescent dye. Non-limiting examples for said labels include cyanine dyes such as Cy3 or Cy5, although any other suitable fluorescent dye may likewise be employed. One specific source for such fluorescently labeled monomers is TriLink BioTechnologies, Inc. of San Diego, Calif., USA. However, such fluorescently labeled monomers can be obtained from other sources if desired, or synthesized using methods well known in the art.

Examples for labels that bind other known specific molecules include, but are not limited to antibodies, ferritin, biotin, streptavidin, polyhistidine tag c-myc tag, histidine-tag and hemagglutinin tag. The labels used in the instant invention are used to either provide a detectable signal in proportion to the amount of target in a given sample, or to affect the binding of target/aptamer-dendrimer complexes to other specific molecules in order to partition the complexes from the remaining sample components. To avoid cross-interactions, the use of the same label moiety for labeling the dendrimer and for attaching the dendrimer to aptamers is undesirable.

More specifically, according to certain embodiments, the dendrimer label consists of ferritin. In yet another embodiment, the target-aptamer-dendrimer complex may be immobilized subsequent to contacting the sample with the target-recognition composition. Thus, in a particular embodiment, the method of the invention concerns partitioning by repeated agitation and application of a magnet, immobilization of target-aptamer-ferritin labeled dendrimer complex on magnet and subsequent removal of target.

In yet another alternative embodiment, the method may further comprise partitioning the target-aptamer-dendrimer complex from the sample, performed, for example, by passage over a column. In more specific embodiments, such column may be an avidin column comprised of High Capacity NeutrAvidin Agarose Resin or any similar resin that allows capture of biotinylated aptamer/dendrimer conjugates bound to the target.

According to some embodiments, the invention relates to a method for detecting, trapping, quantitating, removing, inactivating, or activating at least one target compound in or from a substance or any sample thereof, wherein the dendrimers are each attached to more than one label, more specifically, each dendrimer is attached to between about 2-2000 labels, specifically, 10-20 labels, or each dendrimer is attached to between about 20-30 labels, or each dendrimer is attached to between about 30-60 labels, or each dendrimer is attached to between about 60-80 labels, or each dendrimer is attached to between about 80-100 labels, or each dendrimer is attached to between about 100-120 labels, or each dendrimer is attached to between about 120-140 labels, or each dendrimer is attached to between about 140-160 labels, or each dendrimer is attached to between about 160-180 labels, or each dendrimer is attached to between about 180-200 labels, or each dendrimer is attached to between about 200-220 labels, or each dendrimer is attached to between about 220-240 labels, or each dendrimer is attached to between about 240-260 labels, or each dendrimer is attached to between about 260-280 labels, or each dendrimer is attached to between about 280-300 labels, 300-320 labels, or each dendrimer is attached to between about 320-340 labels, or each dendrimer is attached to between about 340-360 labels, or each dendrimer is attached to between about 360-380 labels, or each dendrimer is attached to between about 380-400 labels, or each dendrimer is attached to between about 400-420 labels, or each dendrimer is attached to between about 420-440 labels, or each dendrimer is attached to between about 440-460 labels, or each dendrimer is attached to between about 460-480 labels, or each dendrimer is attached to between about 480-500 labels, or each dendrimer is attached to between about 500-550 labels, or each dendrimer is attached to between about 550-600 labels, or each dendrimer is attached to between about 600-650 labels, or each dendrimer is attached to between about 650-700 labels, or each dendrimer is attached to between about 700-750 labels, or each dendrimer is attached to between about 750-800 labels, or each dendrimer is attached to between about 800-850 labels, or each dendrimer is attached to between about 850-900 labels, or each dendrimer is attached to between about 900-950 labels, or each dendrimer is attached to between about 950-1000 labels, or each dendrimer is attached to between about 1000-1050 labels, or each dendrimer is attached to between about 1050-1100 labels, or each dendrimer is attached to between about 1100-1150 labels, or each dendrimer is attached to between about 1150-1200 labels, or each dendrimer is attached to between about 1200-1250 labels. More specifically, each dendrimer is attached to between about 10-500 labels. Most specifically, each dendrimer is attached to between about 50-350 labels. In such a manner, each dendrimer emits an amplified detectable signal or is easily bound by partitioning moieties or specific solid supports.

In still yet further specific embodiments, each dendrimer may be attached to between about 100 to about 500 labels. More specifically, each dendrimer may be attached to about 100, 110, 120, 130, 140, 150, 160, 170, 180, 190, 200, 210, 220, 230, 240, 350, 260, 270, 280, 290, 300, 310, 320, 330, 340, 350, 360, 370, 380, 390, 400, 410, 420, 430, 440, 450, 460, 470, 480, 490, 500 labels or even more. Specific embodiment relate to dendrimers attached to about 120 labels. Another specific embodiment relates to a dendrimer attached to about 350 labels.

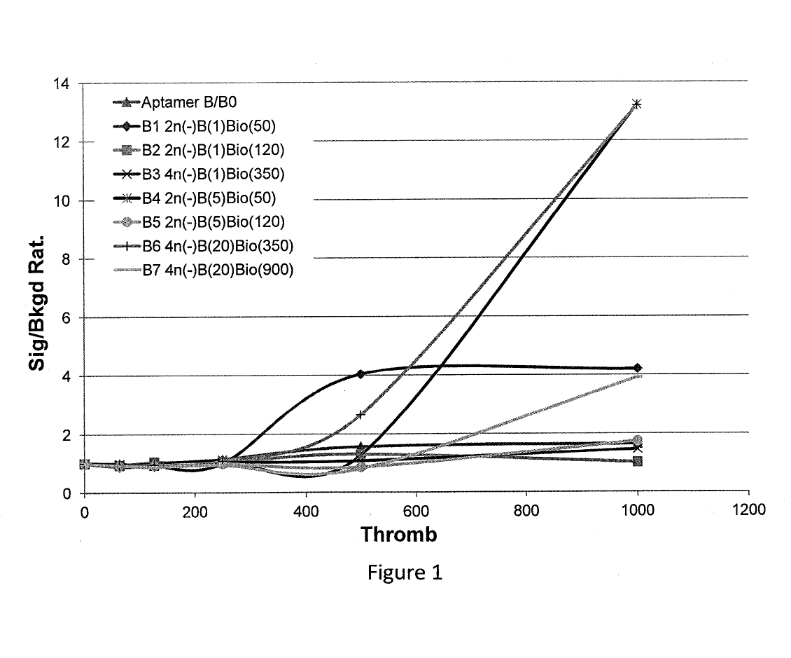

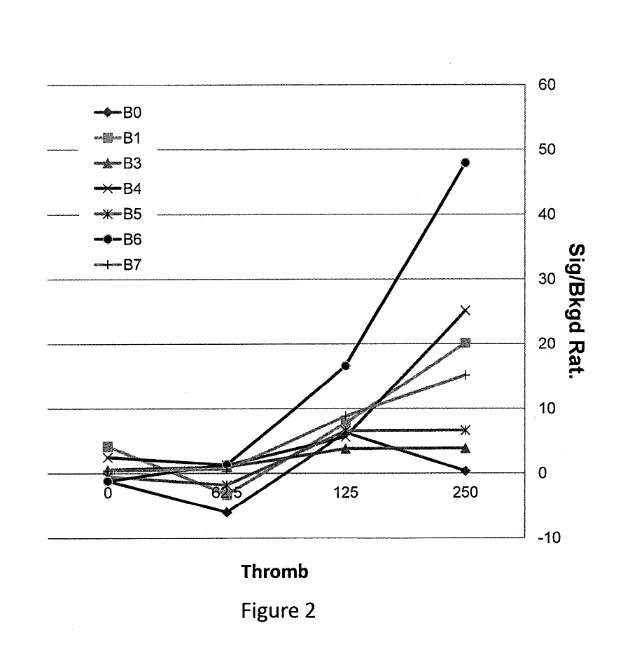

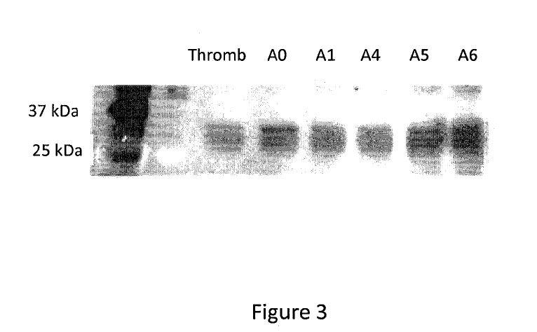

According to yet further specific embodiments, the dendrimers of the invention are particularly effective when comprising a combination of 5 aptamers and 50 labels, or about 20 to about 30 aptamers and 350 labels. A dendrimer attached to one aptamer and 50 labels and a dendrimer attached to about 20 to about 30 aptamers and 900 labels may also be especially effective, as demonstrated in Example 3 and FIGS. 1 and 2. In yet more specific embodiments, the target-recognition composition of the invention may comprise a DNA dendrimer composed of four layers, attached to between about 20-30 aptamers, and labeled with about 350 labeling moieties.

More particular embodiments concern the use of four layer dendrimers attached to between about 20-30 aptamers specific for thrombin, and labeled with about 350 biotin labels. In yet more specific but however, non-limiting embodiments, the thrombin-specific aptamers used by the target-recognition composition of the invention may comprise the nucleic acid sequence of any one of SEQ ID NO. 1 or 2, or of the same sequences having an additional 3′ tail of seven nucleotides as denoted for example by any one of SEQ ID NO. 4 or 5.

In particular embodiments, the invention contemplates a method for detecting, trapping, quantitating, removing, inactivating, or activating at least one target compound in or from a substance or any sample thereof, wherein the detecting occurs on, or involves the use of, a solid support. A solid support suitable for use in the methods and kits of the present invention is typically substantially insoluble in liquid phases. Solid supports of the current invention are not limited to a specific type of support. Rather, a large number of supports are available and are known to one of ordinary skill in the art. Thus, useful solid supports include solid and semi-solid matrixes, such as aerogels and hydrogels, resins, beads, biochips (including thin film coated biochips), microfluidic chip, a silicon chip, multi-well plates (also referred to as microtitre plates or microplates), membranes, filters, conducting and nonconducting metals, glass (including microscope slides) and magnetic supports. More specific examples of useful solid supports include, silica gels, polymeric membranes such as nitrocellulose, particles, derivatized plastic films, glass beads, cotton, plastic beads, alumina gels, polysaccharides such as Sepharose, nylon, latex bead, magnetic bead, paramagnetic bead, superparamagnetic bead, starch and the like.

In certain embodiments of the method of the invention, the target molecule may be any target, for example: polypeptide, oligopeptide, peptide, nucleic acid, deoxyribonucleic acid, ribonucleic acid, glycoprotein, lipoprotein, glycolipoprotein, toxin, carbohydrate, polysaccharides, lipid, a small molecule, a small organic molecule and a non-organic molecule, peptidoglycans, hormones, receptors, antigens, antibodies, viruses, substrates, metabolites, transition state analogs, cofactors, inhibitors, drugs, dyes, nutrients, growth factors, etc. without any limitation.

In other embodiments, where decontamination or purification of a substance from a specific contaminating compound is desired, the method for trapping such target compound as provided by the invention may be particularly useful. In some specific embodiments, the target compound contaminating different substances, for example, environmental substances or water may be, but is not limited to, chemical contaminants, allergens, or drugs-of-abuse.

By “chemical contaminants”, the invention encompasses pesticides, toxins (such as mycotoxins, fungal, bacterial and algal toxins, etc.), veterinary residues (by which is meant any residue that is administered to or consumed by animals that are used for feeding, such as antibiotics and hormones and anti-worm agents such as nitrofuran, fluoroquinolones, malachite green and chloramphenicol), industrial chemicals (including lubricants, hydrocarbons such as polychlorinated biphenyls PCBs, alkylphenols, bisphenol A, triclosan and surfactants, such as nonyl phenol, alkyl ethoxylate and alklyphenol ethoxylate, linear alkylbenzene sulfonate, fire retardants and plasticizers, such as phthalate ester), environmental contaminants (including persistent organic pollutants such as dioxins and contaminants related to water treatment or fertilization, such as copper algaecide, algal herbicide food processing and packaging contaminants (such as nitrosamines, polycyclic aromatic hydrocarbons (PAH), acrylamide, monochloropropanediol (MCPD) heterocyclic amines, histamine, furan, benzene, trans fat, semicarbazide, 4-hydroxynonenal (4-HNE), ethyl carbamate in fermented foods, perchlorate, perfluorooctanoic acid (PFOA), ethyl carbonate, advanced glycation end products (AGEs), perfluorooctanoic acid (PFOA), semicarbazide, benzophenone, isopropylthioxanthone (ITX), and adulterants (such as melamine and casein) as well as cooking equipment cleaning and sanitizing agents, residues of pharmaceuticals and personal care products, poisons, and small molecules (such as arsenic, copper, lead, cadmium and mercury).

In some further specific and non-limiting embodiments, the target compound according to the invention may be naturally occurring toxins. Non limiting examples of such toxins are mycotoxins (including but not limited to fumonisin, patulin, deoxynivalenol, zearalenone, fumonisins B1 and B2 and T-2 and HT-2, patulin, ochratoxin A), bacterial toxins (including but not limited do Botulinum neurotoxin, Tetanus toxin, Anthrax toxin and Staphylococcal toxins) and shellfish toxins.

In other specific embodiments, the target according to the invention may be a pesticide. Pesticides are substances or a mixture of substances intended for preventing, destroying, repelling or mitigating of any pest and generally refers to herbicides, insecticides, fungicides, rodenticides, etc.

In some specific embodiments, the target compound according to the invention may be an herbicide. A herbicide, also known as a weed killer, is a pesticide type used to kill unwanted plants. Selective herbicides kill specific unwanted plants while leaving the desired crop relatively unharmed. Some of these act by interfering with the growth of the weed and are often synthetic imitations of plant hormones. Some non limiting examples of herbicides types include, but are not limited to Acetyl coenzyme A carboxylase (ACCase) inhibitors, acetolactate synthase (ALS) inhibitors, enolpyruvylshikimate 3-phosphate synthase enzyme (EPSPS) inhibitors, Photosystem II inhibitors, etc.

More specifically, examples of herbicides include, but are not limited to: 2,4-D, atrazine, clopyralid, dimethyl tetrachloroterephthalate, dicamba, dichlobenil, diuron, glufosinate, glyphosate, imazapic, Mecoprop (MCPP), oryzalin, pentachlorophenol, picloram, triclopyr and herbicides of the imidazolinone family, such as imazapyr, the sulfonylurea family, such as sulfometuron methyl, tralkoxydim, quizalofop, diclofop, clodinafop, sethoxydim, fenoxyprop, clethodim, difenzoquat, triallate, pendimethalin, trifluralin, ethalfluralin, imazamethabenz, sulfesulfuron, flucarbazone, metsulfuron, triasulfuron, tribenuron, chlorsulfuron, thifensulfuron, prosulfuron, imazapic, imazathapyr, imazamox, sulfosate, paraquat, quinclorac, fluoxypyr, picloram, pyridate and bromoxynil.

In some specific embodiments, the target compound according to the invention may be an insecticide. An insecticide is a pesticide used against insects. Insecticides include ovicides and larvicides, used against the eggs and larvae of insects, respectively. Insecticides are used in agriculture, medicine, industry and in household. Some non-limiting examples of insecticides are systemic insecticides, which are incorporated by treated plants, contact insecticides that are toxic to insects brought into direct contact, natural insecticides, such as nicotine and pyrethrum, Plant-Incorporated Protectants (PIP) that are insecticidal substances produced by plants after genetic modification, inorganic insecticides that are manufactured with metals and include arsenates, copper compounds and fluorine compounds, organic insecticides, etc. Insecticides include insecticides of the carbamate family such as aldicarb and carbaryl, insecticides of the organophosphate family such as chlorpyrifos, diazinon, malathion, naled, synthetic pyrethroids such as cyfluthrin, cypermethrin, permethrin, pyrethrin, resmethrin and sumithrin and other insecticides such as boric acid and borates, chromate copper arsenate, DEBT, dichloropropene, fipronil, imidacloprid (a systemic insecticide), metam sodium, piperonlybutoxide and sulfuryl fluoride.

The target compound according to the invention may be a fertilization agent. The term “fertilizer” relates to any organic or inorganic material of natural or synthetic origin (other than liming materials) that is added to a soil to supply one or more plant nutrients essential to the growth of plants. Fertilization agents include, but are not limited to mined inorganic fertilizers, chemically synthesized inorganic fertilizers, organic fertilizers (e.g. manure, worm castings, compost, seaweed, guano), etc. Specific non-limiting examples for fertilizers include: Ammonium Nitrate 33.5% N, Ammonium 21% N, Ammonium Sulfate Nitrate 26% N, Calcium Ammonium Nitrate (CAN) 27%, Calcium Nitrate 15.5% N, Sodium Nitrate (Natural Chilesalpeter) 16% N, Urea 46% N, Low Biuret Urea, Basic Slag 10% P205, Rock Phosphate 30/32% P205, Single Super phosphate 18/20%) P205 (powder/granular), Triple Super phosphate 46% P205 granular 43% (water soluble), Muriate of Potash 60% K20, Sulphate of Potash 48/52% K20, Diammonium Phosphate (DAP) 18-46-0, Mono Ammonium Phosphate (MAP) 12-52-0 and others.

In some specific embodiments, the target according to the invention may be a poison. Some non limiting examples of poisons include opioids, iron, benzodiazepines, ethylene glycol, methanol, cyanide, organophosphates, calcium channel blockers (e.g. verapamil), arsenic, etc.

The target compound according to the invention may also be a chemical used in the treatment process for water purification, coagulation or disinfection, or that arise from the corrosion products of pipes or that is a degraded form of one of the above. Non limiting examples of chemicals used as water disinfectants are chlorine, chlorine dioxide, monochloramine, granular activated carbon and ozone.

It should be noted that the aptamers suitable for use in the methods, compositions and devices of the invention may be aptamers designed to recognize a variety of targets. More specifically, it must be recognized that the aptamer applicable for the present invention may be aptamers designated to recognize any of the targets disclosed as non-limiting examples herein before. According to the invention, aptamers may be generated to recognize representatives of many of the target classes mentioned. Non-restrictive samples of these include veterinary residues, small molecules as well as metal ions, drugs of abuse, toxins, pesticides, personal care products (including human pharmaceuticals, fragrances, etc.) and industrial contaminants, etc.

In some specific but non-limiting embodiments, aptamers applicable for the invention may be generated to recognize veterinary residues. By the term “veterinary residues” it is referred to any residue that is administered to or consumed by animals that are used for feeding. Non limiting examples of known aptamers developed against veterinary residues include, but are not limited to, aptamers developed against antibiotics (e.g. kanamycin, neomycin B and tobramycin. It should be further noted that aptamers specific for vaccines (e.g. antibodies and antigenic peptides) may be also applicable herein.

In other specific embodiments, aptamers suitable for use by the invention may be aptamers generated to recognize small molecules and metal ions. Examples of aptamers generated to recognize small molecules and metal ions encompassed by the present invention are aptamers designed to bind ethanolamine, mercury ions, potassium ions, or zinc ions.

It is also encompassed by the present invention that aptamers designed against drugs of abuse, such as cocaine may be used by the invention. According to such embodiment, the target recognition composition of the invention may be particularly useful for detection and/or quantification purpose.

In other specific embodiments, aptamers applicable for the present invention may be aptamers generated to recognize toxins. Encompassed by the present invention are non limiting examples for aptamers designed against ochratoxin, botulinum neurotoxin and ricin toxin.

In further specific embodiments, aptamers suitable for the invention may be aptamers generated to recognize pesticides. Encompassed by the present invention are aptamers which recognize acetamiprid, malachite green and organophosphorus pesticides.

In further specific embodiments, aptamers applicable for the invention may be aptamers generated to recognize personal care products (including hormones and other pharmaceuticals, fragrances, etc.). Encompassed by the present invention are aptamers which recognize a human growth hormone, insulin and thyroxine hormone.

In a specific embodiment, the invention encompasses the use of an aptamer that has been developed to recognize thrombin, as exemplified by the present disclosure.

It must be understood that different aptamers that may be applicable for the invention may be prepared and selected by any technique known in the art, including but non limited to SELEX, ASExp or NECEEM or any modifications thereof.

According to other embodiments, the method of the invention is suitable for detecting a target compound in any substance or any sample thereof. The terms “substance”, “sample”, “test sample” and “specimen” are used interchangeably in the present specification and claims and are used in its broadest sense. They are meant to include both biological and environmental samples and may include an exemplar of synthetic origin. This term refers to any media that may contain the target compound and may include body fluids (urine, blood, milk, cerebrospinal fluid, rinse fluid obtained from wash of body cavities, phlegm, pus), samples taken from various body regions (throat, vagina, ear, eye, skin, sores), food products (both solids and fluids) and swabs taken from medicinal instruments, apparatus, materials), as well as substances in which controlled chemical reactions are being carried out.

Biological samples may be provided from animal, including human, fluid, solid (e.g., stool) or tissue, as well as liquid and solid food and feed products, food designed for human consumption, a sample including food designed for animal consumption, food matrices and ingredients such as dairy items, vegetables, meat and meat by-products, and waste. Biological samples and specimens may be obtained from all of the various families of domestic animals, as well as feral or wild animals, including, but not limited to, such animals as ungulates, bear, fish, lagamorphs, rodents, etc. Environmental samples include environmental material such as surface matter, earth, soil, water, air and industrial samples, as well as samples obtained from food and dairy processing instruments, apparatus, equipment, utensils, disposable and non-disposable items. These examples are not to be construed as limiting the sample types applicable to the present invention. The sample may be any media, specifically, a liquid media that may contain the target compound. Typically substances and samples or specimens that are a priori not liquid may be contacted with a liquid media which is contacted with the target recognition composition of the invention.

More specifically, by the term “food”, it is referred to any substance consumed, usually of plant or animal origin. Some non limiting examples of animals used for feeding are cows, pigs, poultry, etc. The term food also comprises products derived from animals, such as, but not limited to, milk and food products derived from milk, eggs, meat, etc.

In some specific embodiments, the present invention encompasses a substance, which is used as a drink. A drink or beverage is a liquid which is specifically prepared for human consumption. Non limiting examples of drinks include, but are not limited to water, milk, alcoholic and non-alcoholic beverages, soft drinks, fruit extracts, etc.

It should be appreciated that in certain cases the substance or any sample thereof can be used in the trapping, removal or detection procedures according to the invention, as originally obtained, without any pretreatment. In many cases, however, the substance or sample should be extracted or lysed using an appropriate method, releasing the target compound contained in therein. Procedures for lysing or extracting substances or samples are known by the expert and can be chemical, enzymatic or physical in nature. A combination of these procedures is applicable as well. For instance, lysis can be performed using ultrasound, high pressure, by shear forces, using alkali, detergents or chaotropic saline solutions, or by means of proteinases or lipases.

The term “extract” as used herein refers to a product prepared by separating, by chemical or physical process. More specifically, “extracts” refers to any substances obtained by extracting the examined substance or any sample thereof using for example, organic solvents such as, ethyl-acetate or hexane.

Reflecting the promising results demonstrated by the enhanced ELISA reaction as shown in Examples 1-4 as well as the efficient contaminant trapping and removal demonstrated by Examples 5 and 6, in the second aspect, the present invention provides a kit for trapping, removing, detecting, quantitating, inactivating, or activating at least one target compound in/or from a substance, or any preparation or sample thereof. In certain embodiments, the kit of the invention may comprise:

(a) at least one nucleic acid based aptamer that specifically recognizes and binds at least one target compound, or complex thereof, or any parts or fragments thereof, or any combinations thereof; and (b) at least one nucleic acid based dendrimer labeled with at least one trapping, partitioning or detectable moiety. In certain embodiments, the kit of the invention may further comprise instructions for carrying out the detection or trapping of said at least one detectable or trapping moiety in said labeled dendrimer. In certain specific embodiments, the kit of the invention may further comprise at least one compartment containing at least one of the aptamer of (a) or the dendrimers of (b) or any target-recognition compositions created by the combination of both (a and b). In yet some optional and particular embodiments, the kit of the invention may optionally comprise an array, wherein each of the dendrimers of (b) may be located in a defined and predetermined position in the array. In further embodiments, the kit of the invention may further comprise at least one assay reagent for enabling the detection or trapping of the at least one detectable or trapping moiety in the at least one labeled dendrimer.

Optionally, the kit of the invention may further comprise instructions for carrying out the detection or trapping of the at least one detectable or trapping moiety in the labeled dendrimer, that binds at least one aptamer in a target-aptamer complex.

In yet another optional embodiment, the kit of the invention may further comprise, pre-determined amount of the at least one target.

In certain embodiments, the kit of the invention may comprise a standard curve indicating predetermined amounts of the target compound of the invention.

It must be understood, that the aptamers and the dendrimers provided as elements (a) and (b) by the kit of the invention may be connected or conjugated to form a target recognition composition.

Targets that may be applicable for the kit of the invention can be a proteins, peptides, carbohydrates, polysaccharides, glycoproteins, peptidoglycans, a variety of small molecules, hormones, receptors, antigens, antibodies, viruses, substrates, metabolites, transition state analogs, cofactors, inhibitors, drugs, dyes, nutrients, growth factors, etc. without limitation. It should be further appreciated that any of the targets described herein above for the compositions and methods of the invention may be also applicable for any of the kits provided by the invention.

It should be noted that the kit of the invention may particularly be suitable for trapping, partitioning and removing a target compound, as for example, the removal of an undesired element from a substance or a sample, or alternatively, an enrichment of a desired element from a substance or sample.

Although such kit may be used also for detection and quantification of a specific target compound, in such diagnostic applications an additional element, such as capture composition may facilitate the detection. Thus, according to other embodiments, the invention provide another version of a kit that may be particularly suited for detecting or quantification at least one target compound in a substance or any sample thereof. In certain embodiments, such kit may further comprise:

At least one capture-composition (d) comprising at least one nucleic acid based or amino acid-based recognition element that specifically recognizes and binds at least one of the target compound or any parts or fragments thereof, or any combinations or complex thereof. It should be noted that the at least one capture-composition is attached to a solid support or a partitioning element.

In certain embodiments, the kit of the invention may further comprise as a fifth element (e), at least one compartment containing at least one of the capture-compositions of (d). In an optional embodiment, the kit may further comprise an array, wherein each of the capture-compositions is located in a defined and predetermined position in the array. According to such embodiment, the examined sample or substance may be applied on the array, before or after being contacted with the aptamer and dendrimers or any combinations thereof as provided by the kit of the invention.

The term “array” as used by the methods and kits of the invention refers to an “addressed” spatial arrangement of the capture composition, or optionally, the target recognition composition. Each “address” of the array is a predetermined specific spatial region containing said capture composition. For example, an array may be a plurality of vessels (test tubes), plates (or even different predetermined locations in one plate or one slide), micro-wells in a micro-plate each containing a different detecting molecule, lanes on a nitrocellulose filter. An array may also be any solid support holding in distinct regions (dots, lines, columns) different detecting molecules. The array preferably includes built-in appropriate controls, for example, regions without the sample, regions without any capture compositions, regions without either, namely with solvent and reagents alone.

The kit of the invention may optionally further comprise instructions for carrying out the detection or quantification of the at least one detectable moiety in the labeled dendrimer provided with the kit of the invention (b), that may be in certain embodiments bound to the at least one aptamer of (a) forming an dendrimers-aptamer-target compound-capture-composition complex attached to a solid support or a partitioning element.

In certain embodiments, the kits of the invention may further comprise means for obtaining a substance or any preparation or sample thereof. It is understood that such substance, tissue or sample used in conjunction with the kit may be any one of a sample drawn from food designed for human consumption, a sample including food designed for animal consumption, food matrices, a water sample, an earth sample, any environmental sample, any cell, tissue, organ or fluid from a biological source, a biological sample drawn from a human patient, a sample drawn from an animal (such as blood, urine, hair) or an organ donation sample.

The kit of the invention is also contemplated wherein its target molecule may be selected from polypeptide, oligopeptide, peptide, nucleic acid, deoxyribonucleic acid, ribonucleic acid, glycoprotein, lipoprotein, glycolipoprotein, toxin, carbohydrate, lipid, a small molecule, a small organic molecule and a non-organic molecule.

According to specific embodiments, the kit of the invention is for performing the method according to the invention.

In some embodiments, the kit for detecting or quantification at least one target compound in a substance or any sample thereof according to the invention is contemplated. In other embodiments, the kit is for trapping at least one target compound in a substance or any sample thereof. In further embodiments, the kit is for removing at least one target compound away from a substance or any sample thereof. Still further, the kit of the invention may be used for enrichment and purification of a target compound. Yet further embodiments consider this kit for inactivating at least one target compound in a substance or tissue or any sample thereof.

In various embodiments of the kit of the invention, the nucleic acid based or amino-acid-based recognition element comprised within the capture-composition may be any one of a nucleic acid selected from: aptamer, probe, primer, or oligonucleotide specific for the target, or an amino-acid selected from: an antibody, peptide, ligand, receptor or substrate, or any small molecule specific for the target.

It is appreciated that the aptamer and dendrimer provided by the kit of the invention may be attached or conjugated prior to contacting both with the substance or any sample thereof, alternatively, they may be attached simultaneous with or after contacting both with the substance or any sample thereof.

In certain embodiments, the kit of the invention is contemplated, wherein each dendrimer specifically recognizes and binds a specific aptamer or a specific aptamer-target complex.

According to some embodiments, the dendrimers may be comprised of deoxyribonucleic acids.

It can be understood that the dendrimers may be either directly attached to the aptamers, or attached to the aptamers via nucleic acid complementarity. The attachment of the dendrimers to the aptamers via nucleic acid complementarity may be followed by cross linking. Other embodiments consider the attachment of the dendrimers to the aptamers via a bridging complementary nucleic acid followed by ligation, or by cross linking, such as UV cross linking.

According to other embodiments, the dendrimers may also be attached to the aptamers via a biological affinity system.

In various embodiments, the aptamer-dendrimer combinations according to the kit of the invention may take different forms; the dendrimers may each be attached to or conjugated with more than one identical or different aptamers.

In particular embodiments, the invention relates to a kit for detecting, trapping, quantitating, removing, inactivating, or activating at least one target compound in or from a substance or any sample thereof, wherein the dendrimers are each attached to more than one aptamer, more specifically, each dendrimer is attached to between about 1-50 aptamers, specifically, 2-5 aptamers, or each dendrimer is attached to between about 6-9 aptamers, or each dendrimer is attached to between about 10-13 aptamers, or each dendrimer is attached to between about 14-17 aptamers, or each dendrimer is attached to between about 18-21 aptamers, or each dendrimer is attached to between about 20-30 aptamers, or each dendrimer is attached to between about 22-25 aptamers, or each dendrimer is attached to between about 26-29 aptamers, or each dendrimer is attached to between about 30-33 aptamers, or each dendrimer is attached to between about 34-37 aptamers, or each dendrimer is attached to between about 38-41 aptamers, or each dendrimer is attached to between about 42-45 aptamers, or each dendrimer is attached to between about 46-49 aptamers, or each dendrimer is attached to between about 50-53 aptamers. Particularly useful for the kit are dendrimers which are attached to between about 5-30 aptamers. In such a manner, multimeric and/or multivalent aptamer ligands are presented to a single or multiple target molecules.

Furthermore, the aptamers bound to a dendrimer may be different aptamers recognizing different or identical targets, or different aptamers that recognize identical targets. They may also be different aptamers that recognize different targets.

Specific embodiments relate to a kit providing a dendrimer comprising between about 20 to 30 aptamers and about 350 labeling moieties

Some aptamer-dendrimer configurations contemplated by the embodiments of the kit of the invention are particularly advantageous for trapping, removing or inactivating of one or more targets in a sample. According to these embodiments, the target-recognition composition comprises at least one dendrimer and at least one aptamer, and the aptamers may be different aptamers recognizing different targets. The trapping, removing or inactivating is of at least one target compound in or from a substance or tissue or any sample thereof.

In particular embodiments of the kit of the invention, the detection (or detection combined with trapping) step further comprises the contacting of the sample with a capture-composition that recognizes a specific aptamer-dendrimer-target complex, whereas in other embodiments the contacting step comprises the contacting of the sample with a capture-composition that recognizes a specific target prior to the contacting of sample with the aptamer or any target recognition composition comprising a combination or conjugate of both aptamer and dendrimer.

Further embodiment consider the kit of the invention, wherein the composition comprises at least two dendrimers, each of the dendrimers is labeled with at least one identical detectable and/or trapping and/or partitioning moiety, and wherein each of the dendrimers is attached or conjugated to at least one identical or different aptamer, the aptamers recognizing an identical target. In more specific embodiments, each different target is recognized by aptamers attached to differently-labeled dendrimers.

Whatever the specific configuration of the target-recognition composition in terms of aptamers and labels per dendrimer, the instant invention provides a greatly enhanced specific and sensitive system for detecting, trapping, quantitating, removing or inactivating at least one target compound in or from a substance or tissue or any sample thereof. In the instant invention, each aptamer is specific to a molecular target or targets, and the dendrimer carrying at least one aptamer and at least one label, and specifically, more than one label, enhances the sensitivity of the target detection. The conjugated or attached aptamer-aptamer-dendrimer is therefore both sensitive and specific, and is being claimed here.

The term “sensitivity” is used herein with respect to the ability of a specific aptamer, or a aptamer-dendrimer, to bind the intended target of said aptamer or aptamer-dendrimer even when said target is present in a low concentration in a sample, said concentration may be less than 100 mM, less than 50 mM, less than 10 mM, less than 1 mM, less than 800 μM, less than 600 μM, less than 500 μM, less than 400 μM, less than 300 μM, less than 200 μM, less than 100 μM, less than 50 μM, less than 10 μM, less than 1 μM, less than 900 nM, less than 800 nM, less than 700 nM, less than 600 nM, less than 500 nM, less than 400 nM, less than 300 nM, less than 200 nM, less than 100 nM, less than 500 nM, less than 1000 nM, less than 1 nM, less than 900 pM, less than 800 pM, less than 700 pM, less than 600 pM, less than 500 pM, less than 400 pM, less than 300 pM, less than 200 pM, less than 100 pM, less than 50 pM, less than 1 pM, less than 5 pM.

The term “specificity” is used herein with respect to the ability of a specific aptamer, or a aptamer-dendrimer, to preferentially bind the intended target of said aptamer or aptamer-dendrimer in a sample, said aptamer, or a aptamer-dendrimer binds the intended target at least 1.5 fold more than any other ingredient in said sample, at least 2.0 fold, at least 2.5 fold, at least 5.0 fold, at least 10.0 fold, at least 20.0 fold, at least 50.0 fold, at least 100 fold, at least 200 fold, at least 500 fold, at least 1000 fold, at least 5000 fold, at least 10000 fold, at least 50,000 fold, or at least 100,000 fold.

The inventors consider the kit of the invention in compartmental form. In yet another embodiment, the kit comprising a compartment adapted to contain one or more arrays. For example, an array may contain different target-recognition compositions obtained by combination of the aptamers and dendrimers provided by the kit of the invention, different capture-compositions, or both. As indicated herein before, the different target-recognition compositions or the different capture-compositions may be spatially arranged in a predetermined and separated location in the array. It is not overlooked that in embodiments where the number of different targets is small and can be conveniently differentiated by differential labeling, e.g., by FITC versus rhodamine, spatial segregation for multiplexing will not be necessary. For example, an array may be a plurality of vessels (test tubes), plates, micro-wells in a micro-plate lanes or dots in a nitrocellulose strip, each containing a different target-recognition composition or a capture-composition. An array may also be any solid support holding in distinct regions (dots, lines, columns) different and known, predetermined target-recognition composition or a capture-composition.

As used herein, “solid support” is defined as any surface to which molecules may be attached through either covalent or non-covalent bonds. Thus, useful solid supports include solid and semi-solid matrixes, such as aerogels and hydrogels, resins, beads, biochips (including thin film coated biochips), microfluidic chip, a silicon chip, multi-well plates (also referred to as microtiter plates or microplates), membranes, filters, conducting and nonconducting metals, glass (including microscope slides) and magnetic supports. More specific examples of useful solid supports include silica gels, polymeric membrane such as nitrocellulose, particles, derivatized plastic films, glass beads, cotton, plastic beads, alumina gels, polysaccharides such as Sepharose, nylon, latex bead, magnetic bead, paramagnetic bead, superparamagnetic bead, starch and the like. This also includes, but is not limited to, microsphere particles such as Lumavidin™ or LS-beads, magnetic beads, charged paper, Langmuir-Bodgett films, functionalized glass, germanium, silicon, PTFE, polystyrene, gallium arsenide, gold, and silver. Any other material known in the art that is capable of having functional groups such as amino, carboxyl, thiol or hydroxyl incorporated on its surface, is also contemplated. This includes surfaces with any topology, including, but not limited to, spherical surfaces and grooved surfaces.

It should be further noted that any of the reagents included in any of the methods and kits of the invention may be provided as reagents embedded, linked, connected, attached, placed or fused to any of the solid support materials described above.

In a third aspect, the invention relates to a device for trapping, removing, detecting, quantitating, inactivating, or activating at least one target compound in/or from a substance or any preparation or sample thereof, comprising:

a. at least one target-recognition composition comprising at least one nucleic acid based aptamer that specifically recognizes and binds the at least one target compound, or complex thereof, or any parts or fragments thereof, or any combinations thereof, and at least one nucleic acid based dendrimer labeled with at least one detectable and/or trapping and/or partitioning moiety; and
b. a solid support suitable for the reception and transport of the sample.

Such devices for detection and quantification of target compounds may be particularly suited for commercial uses in easy to use formats, such as lateral flow system. Thus, in certain embodiments, the present invention provides a method for the detection and analysis of analytes via a lateral flow assay system. By “lateral flow” it is meant that a sample suspected of containing an analyte is placed on a test strip consisting of a bibulous, chromatographic or other porous material and the sample is wicked laterally through of the test strip by capillary action, coincidentally reacting with various reagents in the strip. The scope of the invention is not limited with respect to the direction of the sample movement through the test strip.

Lateral flow tests are devices intended to detect the presence (or absence) of a target analyte in a sample (matrix). Many commonly used lateral flow tests are for medical diagnostics either for home testing, point of care testing, or laboratory use. Some are for environmental and food testing. Often produced in a dipstick format, lateral flow tests are a form of immunoassay in which the test sample flows along a solid porous substrate via capillary action. In some cases, after the sample is applied to the test it encounters a colored reagent which mixes with the sample and transits with it in the substrate, encountering lines or zones which have been pretreated with a capturing molecule. In the instant invention, the colored reagent may be the same or different aptamers arranged on a dendrimer scaffold labeled with a colored or otherwise detectable label. Depending upon the target analytes present in the sample, the colored reagent (labeled aptamer/dendrimers) can become bound at the test line or zone. The test line will show as a colored band or spot in positive samples. Most tests are intended to operate on a purely qualitative basis. However it is possible to measure the intensity of the test line to determine the quantity of analyte in the sample. Handheld diagnostic devices known as lateral flow readers are used by several companies to provide a fully quantitative assay result. By utilizing unique wavelengths of light for illumination in conjunction with either CMOS or CCD detection technology, a signal rich image can be produced of the actual test lines. Using image processing algorithms specifically designed for a particular test type and medium, line intensities can then be correlated with analyte concentrations. One such handheld lateral flow device platform is made by Detekt Biomedical L.L.C., Alternative non-optical techniques are also able to report quantitative assays results. One such example is a magnetic immunoassay (MIA) which, in the lateral flow test form, also allows for getting a quantified result. One may also obtain semi-quantitative result by comparison of signals emitted by the aptamer-dendrimer to the intensity of signal observed in a calibration curve, or with any known amount.

For labeling of said lateral flow assays, in principle, any colored particle can be used, however commonly either latex (blue color) or nanometer sized particles of gold (red color) are used. Fluorescent or magnetic labeled particles can also be used, however, these require the use of an electronic reader to assess the test result.

Lateral flow Tests can operate as either direct or competitive sandwich assays.

According to particular embodiments, the device according to the invention is especially suited to performing the method according to the invention.

In another embodiment of the invention, a device for detecting or quantification of at least one target compound in a substance or any sample thereof using a lateral flow assay, may comprise:

a. a solid support suitable for the reception and transport of the sample;
b. at least one target-recognition composition comprising at least one nucleic acid based aptamer that specifically recognizes and binds the at least one target compound, or complex thereof, or any parts or fragments thereof, or any combinations thereof, and at least one nucleic acid based dendrimer labeled with at least one detectable moiety. The at least one target-recognition composition is located in a predetermined specific initiation zone in the solid support.
c. at least one capture-composition comprising at least one nucleic acid based or amino acid-based recognition element that specifically recognizes and binds at least one of the target compound or any complex thereof. The at least one capture-composition may be attached to the solid support in a predetermined location in an termination zone in the solid support.

In an additional embodiment, a further indicator means, which indicates that the sample has been transported along the solid support and confirms that the reagent(s) are operable, may be further added.

The solid support may be a porous inert material selected from paper, nitrocellulose glass, nylon and plastic.

According to certain embodiments, an elongated rectangular component, often of paper, nitrocellulose or other porous inert material, upon which are printed stripes or layers of assay reagents is being used as a solid support.

Of course, to facilitate or enhance the detection of the target, the device may further comprise at least one assay reagent for enabling the detection or trapping of the at least one detectable or trapping moiety in the at least one labeled dendrimer. The reagents may be placed, attached, embedded or located, or connected to the solid support.

The contemplated device may be particularly suited to performing the method according to the invention.

In yet a further embodiment, the invention provides a flow-through device for trapping, partitioning or removing at least one target compound from a substance or any preparation or sample thereof. In certain embodiments such device may comprise:

a. a solid support suitable for the reception and transport of the sample comprising a sample port and a flow path extending from the sample port. In certain embodiments, such solid support may be in the form of a cartridge. It should be noted that the flow path includes partitioning-elements for capturing said at least one trapping or partitioning moiety attached to said dendrimer comprised within said target-recognition composition;
b. at least one target-recognition composition provided with the sample port or captured in said flow path by said capturing-elements.

According to such embodiment, the sample placed in the sample port and is forced to flow through the flow path, optionally, into a collection or waste chamber, thereby capturing the analyte attached to the target-recognition composition labeled with a trapping moiety, with the partitioning-elements as the sample flows through the flow path. The captured target analyte may then eluted from the flow path by forcing an elution fluid to flow through the flow path, for optional quantification or purification (after recovery) purpose.

It should be noted that in certain embodiments, the device of the invention may be used for removing or decontaminating an undesired analyte. However, in alternative embodiments the device of the invention may be used for purification or enrichment of the trapped target compound from the substance.

It should be understood that the present invention further encompasses any of the target-recognition compositions used by any of the methods, kits and devices of the invention. More specifically, the invention further provides a target-recognition composition comprising at least one nucleic acid-based aptamer, specifically, a DNA aptamer, that specifically recognizes and binds the at least one target compound, or complex thereof, or any parts or fragments thereof, or any combinations thereof, and at least one nucleic acid based dendrimer, specifically, a DNA dendrimer, labeled with at least one detectable and/or trapping and/or partitioning moiety. In particular specific embodiments, the target-recognition compositions of the invention may comprise at least one DNA aptamer-DNA dendrimer conjugate.

While the invention will now be described in connection with certain preferred embodiments in the following examples so that aspects thereof may be more fully understood and appreciated, it is not intended to limit the invention to these particular embodiments. On the contrary, it is intended to cover all alternatives, modifications and equivalents as may be included within the scope of the invention as defined by the appended claims. Thus, the following examples which include preferred embodiments will serve to illustrate the practice of this invention, it being understood that the particulars shown are by way of example and for purposes of illustrative discussion of preferred embodiments of the present invention only and are presented in the cause of providing what is believed to be the most useful and readily understood description of formulation procedures as well as of the principles and conceptual aspects of the invention.

Therefore, it is to be understood that this invention is not limited to the particular examples, process steps, and materials disclosed herein as such process steps and materials may vary somewhat. It is also to be understood that the terminology used herein is used for the purpose of describing particular embodiments only and not intended to be limiting since the scope of the present invention will be limited only by the appended claims and equivalents thereof.

It must be noted that, as used in this specification and the appended claims, the singular forms “a”, “an” and “the” include plural referents unless the content clearly dictates otherwise.

By “consisting of” is meant including, and limited to, whatever follows the phrase “consisting of”. Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

The term “about” as used herein indicates values that may deviate up to 1%, more specifically 5%, more specifically 10%, more specifically 15%, and in some cases up to 20% higher or lower than the value referred to, the deviation range including integer values, and, if applicable, non-integer values as well, constituting a continuous range.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

Unless defined otherwise, all technical and scientific terms used herein have the same meaning as commonly understood by one of ordinary skill in the art to which this invention is related. The following terms are defined for purposes of the invention as described herein.

In carrying out the present invention, unless otherwise indicated, conventional techniques of chemistry, molecular biology, biochemistry, protein chemistry, and recombinant DNA technology, may be employed, all of which are within the skill of the person skilled in the art.

The following Examples are representative of techniques employed by the inventor in carrying out aspects of the present invention. It should be appreciated that while these techniques are exemplary of preferred embodiments for the practice of the invention, those of skill in the art, in light of the present disclosure, will recognize that numerous modifications can be made without departing from the spirit and intended scope of the invention.

### EXAMPLES

### Materials

Aptamer A, which recognizes thrombin, has the sequence 5′ GGT TGG TGT GGT TGG 3′ (also denoted by SEQ ID NO. 1) and was synthesized by IDT either with a 3′ biotin moiety or with a seven base nucleotide tail at the 3′ end, creating an elongated 22-mer sequence: 5′ GGT TGG TGT GGT TGG TTT TTC G 3′ (as denoted by SEQ ID NO. 4).

Aptamer B, which also recognizes thrombin, has the sequence 5′ GTC CGT GGT AGG GCA GGT TGG GGT GAC 3′ (also denoted by SEQ ID NO. 2) and was synthesized by IDT either with a 3′ biotin moiety or with a seven base nucleotide tail at the 3′ end, creating an elongated 34-mer sequence: 5′ GTC CGT GGT AGG GCA GGT TGG GGT GAC TTT TTC G 3′ (as denoted by SEQ ID NO. 5).

Thrombin (T6884), Streptavidin HRP (E2886), TMB Substrate for ELISA (T0440) and Stop Reagent for TMB Substrate (S5814) were all purchased from Sigma. High Capacity NeutrAvidin Agarose Resin (PIR-29202) of Thermo Scientific was used for pull down studies. The antibodies used were Ab61367 sheep anti-thrombin and Ab7111 HRP-conjugated rabbit anti-sheep, both from Abeam. For gel analysis RunBlue 12% SDS Gel of Expedeon was used (including manufacturer's supplied sample buffer and DTT) and silver staining was carried out with PlusOne Silver Staining Kit for Proteins of GE Healthcare. Direct detection of analyte was carried out following absorption on Nunc MaxiSorb plates. Following color development, plates were read using the FLUOstar OPTIMA reader by BMG Labtech.

### Experimental Procedures

### Biotin Avidin Pull-Down Assay

Thrombin containing solutions (500 ng thrombin) were incubated for 3 hours at RT with shaking, in the presence of 100 ng of biotinylated aptamer alone (A0) or multiply biotinylated aptamer-dendrimer conjugates (A1, A4, A5), or in the absence of any analyte trapping moiety, in 100 μl of PBS. Following this incubation period, 20 μl of High Capacity NeutrAvidin Agarose Resin from ThermoScientific that has been washed to remove the azide present in the PBS storage solution, was added to each of the solutions and incubation was continued (RT, 1 hour). Subsequently, the NeutrAvidin Resin, with all bound biotinylated moieties and their attached target thrombin, was pelleted by brief centrifugation (1-2 minutes, 2,500×g). The supernatants, containing target analyte that has not been trapped, was removed. The thrombin in each of these supernatants was allowed to absorb onto microtitre plates, at 4° C., overnight.

A calibration curve of thrombin was prepared on the same plate, by allowing thrombin-PBS solutions, containing thrombin at 0-500 ng, to adhere at 4° C., overnight. The following day, a comparison was made of the amount of untrapped analyte originating from the supernatants derived from tubes with no trapping moiety, with the aptamer alone and with aptamer-dendrimer conjugates, as well as the calibration curve, by carrying out direct ELISA with commercial antibodies. Briefly, this was done by washing with PBS supplemented with 0.1% Tween 20, blocking with PBS supplemented with 5% non-fat dry milk for 1 hour, and then carrying out the detection, first by exposure to sheep anti-thrombin antibodies (1:500 diluted in 5% nonfat dry milk/PBS) for 1 hour, and then, following washing, by exposure to peroxidase conjugated rabbit anti-sheep secondary antibody (1:10,000 diluted in PBS/0.1% Tween 20) for an additional hour. Following additional washes, color development was achieved with a TMB substrate and stopped with TMB stop solution (Sigma). Absorbance was read at 450 nm.

### SDS-Gel Analysis of Neutravidin Agarose Resin-Bound Thrombin

Neutravidin agarose resin with bound aptamer or aptamer/dendrimer conjugates was prepared as described in the pull down assay above. The resin comprising the associated target recognition aptamer or conjugates and bound thrombin was washed and resuspended in 75 μl PBS. Aliquots (30 μl) were taken from each sample, sample buffer (10 μl) and DTT (4 μl) was added to each, and the aliquots were heated (70° C.) and then loaded onto the 12% SDS gel. SDS gels were treated by silver staining to enhance observation sensitivity.

### Controlled Synthesis of Dendrimers

Dendrimers were synthesized by Genisphere LLC (PA USA). DNA dendrimer size was controlled by varying the number and ratios of layers of various single stranded DNA molecule monomers that were used for synthesizing the polymeric DNA scaffold. Each monomer was made from two DNA strands that share a region of sequence complementarity located in the central portion of each strand. When the two strands anneal to form the monomer the resulting structure can be described as having a central double-stranded “waist” bordered by four single-stranded “arms”. This waist-plus-arms structure comprises the basic DNA monomer. The single-stranded arms at the ends of each of the five monomer types are designed to interact with one another in precise and specific ways. Base-pairing between the arms of complementary monomers allowed directed assembly of the dendrimer through sequential addition of monomer layers. Assembly of each layer of the dendrimer included a cross-linking process where the strands of DNA were covalently bonded to each other, thereby forming a completely covalent molecule impervious to denaturing conditions that otherwise would cause deformation of the dendrimer structure. The number of biotin labels on each polymeric dendrimer was controlled by the ratio of biotin labeled DNA oligonucleotide monomers used during synthesis.

DNA aptamers were conjugated to the dendrimers via a DNA ligation reaction by using a bridging oligonucleotide (5′-TCA CAT ACG ACT CCG AAA AA-3′, also denoted as SEQ ID NO.:3) that spans the ligation site between sequences that it recognizes in the dendrimer single stranded “arm” and the seven (7) oligonucleotide tail that was added to the 3′ end of the aptamers. More specifically, ligation of aptamers A or B (as denoted by SEQ ID NO. 1 and 2, respectively) was performed by a T4 DNA ligase dependent ligation reaction, as follows: About 5.4 uL (2680 ng) of four layer DNA dendrimer (500 ng/uL) in 1×TE buffer, were added to 2.7 uL (134 ng) Bridging oligo (SEQ ID NO. 3) (50 ng/uL) and 10.2 Ul of 10× Ligase buffer in Nuclease free water (81.7 uL). The mixture has been heated to 65° C. and cooled to room temperature. Aptamers A or B DNA oligo (4.0 uL (200 ng) 50 ng/uL) and T4 DNA Ligase (1 U/uL) (10.0 uL (10 units)) were added and incubated for 45 minutes. The ligation reaction was stopped by adding 2.8 uL of 0.5M EDTA solution. Non-ligated oligonucleotide was removed via the use of a size exclusion spin column prepared using Sephacryl S400 (Pharmacia).

The number of aptamers conjugated to each dendrimer was controlled by the ratio of aptamer molecules to dendrimer arms bearing the recognition sequence for the bridging molecule. In the case of the largest dendrimers employed, the dendrimers with 900 biotin labels, single stranded extension DNA molecules were bound to dendrimer arms, providing additional sites for binding an excess of biotin labeled DNA oligonucleotides.

As shown in Table 1 below, for each of the two aptamers, A and B, which recognize thrombin, seven different aptamer-dendrimer conjugates were synthesized. These vary in overall size, in the number of biotin labels and the number of aptamers conjugated to each dendrimer. The number of labels varies between 50 to 900 and the number of aptamers per dendrimer varies from a single aptamer to 20-30 per dendrimer. Thus, including the unconjugated aptamers with no dendrimer attached, each of the aptamer-dendrimer series has eight members.

TABLE 1
Aptamer-dendrimer conjugates used in the examples for each conjugate,
the table describes the number of aptamers bound per dendrimer,
the dendrimer size (number of layers and MW), and the number
of labels on the aptamer-dendrimer conjugate

| Code | Aptamer | Dendrimer | Appr. MW |
|---|---|---|---|
| A0 | Aptamer A | none | 5.2 × 10⁴ |
| | | (one biotin label) | |
| A1 | Aptamer A | 2 layer | 1.8 × 10⁶ |
| | (one per dendrimer) | (50 biotin labels) | |
| A2 | Aptamer A | 2 layer | 4.1 × 10⁶ |
| | (one per dendrimer) | (120 biotin labels) | |
| A3 | Aptamer A | 4 layer | 1.4 × 10⁷ |
| | (one per dendrimer) | (350 biotin labels) | |
| A4 | Aptamer A | 2 layer | 1.8 × 10⁶ |
| | (five per dendrimer) | (50 biotin labels) | |
| A5 | Aptamer A | 2 layer | 4.1 × 10⁶ |
| | (five per dendrimer) | (120 biotin labels) | |
| A6 | Aptamer A | 4 layer | 1.4 × 10⁷ |
| | (20-30 per dendrimer) | (350 biotin labels) | |
| A7 | Aptamer A | 4 layer | 2.2 × 10⁷ |
| | (20-30 per dendrimer) | (900 biotin labels) | |
| B0 | Aptamer B | none | 8.9 × 10⁴ |
| | | (one biotin label) | |
| B1 | Aptamer B | 2 layer | 1.8 × 10⁶ |
| | (one per dendrimer) | (50 biotin labels) | |
| B2 | Aptamer B | 2 layer | 4.1 × 10⁶ |
| | (one per dendrimer) | (120 biotin labels) | |
| B3 | Aptamer B | 4 layer | 1.4 × 10⁷ |
| | (one per dendrimer) | (350 biotin labels) | |
| B4 | Aptamer B | 2 layer | 1.8 × 10⁶ |
| | (five per dendrimer) | (50 biotin labels) | |
| B5 | Aptamer B | 2 layer | 4.1 × 10⁶ |
| | (five per dendrimer) | (120 biotin abels) | |
| B6 | Aptamer B | 4 layer | 1.4 × 10⁷ |
| | (20-30 per dendrimer) | (350 biotin labels) | |
| B7 | Aptamer B | 4 layer | 2.2 × 10⁷ |
| | (20-30 per dendrimer) | (900 biotin labels) | |

Aptamer A is also denoted by SEQ. ID. NO. 1, and Aptamer B is denoted by SEQ ID NO. 2

For each of the two series, the seven aptamer-dendrimer conjugates bearing 50 to 900 biotin labels were compared with the aptamer alone (A0), bearing a single biotin label, with respect to sensitivity of detection of their target analyte and signal amplitude. In additional studies, the aptamer-dendrimer conjugates were compared to aptamer alone with respect to ability to capture and deplete their target analyte from solution.

### Example 1

### Enhanced Sensitivity of Detection by Aptamer-Dendrimer Conjugates of the a Series in Comparison to Aptamer Alone Upon Direct Detection of Thrombin Analyte Absorbed Onto Microtitre Plates at Two Concentrations

The ability of aptamer-dendrimer conjugates (listed in Table 1 hereinabove) to increase the sensitivity of detection relative to aptamer alone was examined. To accomplish this, Aptamer A, which recognizes thrombin, was biotinylated to allow for detection of aptamer binding per se. Binding and signal production of the aptamer A0 was compared to binding by the same unbiotinylated aptamer that is conjugated with each of the seven different biotinylated dendrimer conformations (numbers A1-A7 in the series above). These dendrimer conformations are also shown in Table 2 below. As can be seen, they include conjugates with 1, 5, or 20-30 aptamers complexed per dendrimer, and with 50, 120, 350 or 900 biotin labels. All conjugates between aptamer and dendrimers are generated by ligation mediated by a bridging molecule.

Thrombin was fixed to microtitre wells by exposing wells to 100 μl of solutions containing either 25 or 100 ng of thrombin/100 ul. Absorption of thrombin was demonstrated by antibody-based immunoassay, using commercial antibodies. For comparison of their ability to detect the absorbed thrombin, the biotinylated aptamer A and the seven different biotinylated aptamer-dendrimer conjugates shown in the table below were applied (50 ng/well) to duplicate wells that had been exposed to each thrombin concentration. Following a two hour incubation, the wells containing attached biotinylated aptamer and biotinylated aptamer-dendrimer complexes were washed, exposed first to streptavidin-HRP and then to TMB substrate. The reaction was stopped with TMB stop solution and read at OD 420 nm.

TABLE 2
Aptamer/dendrimer and label number combinations

| Code | Aptamer | Aptamers per Dendrimer | Labels |
|---|---|---|---|
| A0 | Aptamer A | No dendrimer | 1 label |
| A1 | Aptamer A | 1 | 50 labels |
| A2 | Aptamer A | 1 | 120 labels |
| A3 | Aptamer A | 1 | 350 labels |
| A4 | Aptamer A | 5 | 50 labels |
| A5 | Aptamer A | 5 | 120 labels |
| A6 | Aptamer A | 20-30 | 350 labels |
| A7 | Aptamer A | 20-30 | 900 labels |

Following detection of signal, the signal to background ratio was calculated for each aptamer or aptamer-dendrimer conjugate at each thrombin concentration by comparing to the signal observed in wells with thrombin to the signal obtained in the control wells with no thrombin. The results are shown below in Table 3.

TABLE 3
Comparison of thrombin detection by Aptamer A alone
(A0) and by aptamer-dendrimer conjugates of the A series
(A1-A7), shown as signal:background ratio in comparison
with control wells containing no thrombin

| Aptamer conjugate | 100 ng | 25 ng |
|---|---|---|
| A0 | 1.00 | O.95 |
| A1 | 1.11 | 0.98 |
| A2 | 1.08 | 0.95 |
| A3 | 1.19 | 1.11 |
| A4 | 1.07 | 1.03 |
| A5 | 1.08 | 1.02 |
| A6 | 1.11 | 1.13 |
| A7 | 1.04 | 1.04 |

As shown in Table 3, aptamer A alone (A0) is not able to detect the thrombin fixed to the microtiter plate at either thrombin concentration, 25 or 100 ng, since it gives a signal to background ratio less than or equal to 1 at these two concentrations, respectively. By contrast, taking a greater than 5% increase in signal to background ratio as a cut off, six of seven of the A series aptamer-dendrimer conjugates (A1-A6) detect 100 ng of thrombin and display an increase in signal over the control that ranges from 7% to 19%. There is a clear trend towards a higher signal to background ratio among the 6 aptamer-dendrimer conjugates (A1-A6) which on average display an 11% increase compared to aptamer A alone (A0).

Two of the aptamer-dendrimer conjugates, which are also among those, which gave the highest signal to background ratio with 100 ng of thrombin, display even greater sensitivity and are able to detect the presence of 25 ng of thrombin. These two conjugates, displayed an increase in signal to background ratio in the presence of 25 ng of thrombin (11% for A3 and 13.0% for A6).

Since no signal was observed with the aptamer alone (A0) even at 100 ng of thrombin, it is evident that the complexing of aptamers to dendrimers in the aptamer-dendrimer conjugates brings about at least a 4-fold increase in sensitivity. This has been established by the demonstration that dendrimer conjugated aptamers detected 25 ng of the target analyte while the unconjugated aptamer did not reveal the presence of 100 ng. In fact, the increase in sensitivity is likely even higher (an estimated 40 fold for the four conjugates which detect 25 ng of the target analyte and 10 fold for the other three which give a signal over background only when bound to 100 ng of thrombin) as other studies employing Aptamer A alone (A0) have demonstrated thrombin detection by Aptamer A0 under conditions similar to those employed here only when 1000 ng of thrombin was applied to microtitre wells.

While six of the seven different aptamer-dendrimer conjugates conferred on the aptamer increased sensitivity which enabled detection of 100 ng of thrombin, the conjugates displaying the greatest increase in signal were A3 (a 4 layer dendrimer with a single aptamer and 350 biotin labels, which gave an almost 19% increase in signal), A6 (a four layer dendrimer with 20-30 attached aptamers and 350 labels, which gave an about 11% increase in signal) and A1 (a two layer aptamer with a single attached dendrimer and 50 labels which also gave an about 11% increase in signal). The conjugate which did not lead to an increase in signal to background ratio was A7 with 900 biotin labels. It can be deduced from this that not just the number of signal molecules, but also other factors such as steric factors, affect the ability of aptamer-dendrimer conjugates to enhance signal sensitivity.

It has been demonstrated that when aptamers are employed to detect the presence of an analyte (or a target molecule), the sensitivity of detection by the aptamer can be increased by its conjugation with a multiply labeled DNA dendrimer in an aptamer-dendrimer conjugate. Relative to aptamer A alone, sensitivity of detection was enhanced by many, but not all, of the aptamer-dendrimer conjugates with different size dendrimers, numbers of label moieties on the dendrimer and numbers of aptamers incorporated into the dendrimer scaffold. In particular, the highest enhancement in the sensitivity of the detection was obtained when the two aptamer-dendrimer conjugates A3 and A6 were employed, as demonstrated by their ability to detect 25 ng of target. While A3 comprises a 4 layer dendrimer, with 350 labels and a single conjugated aptamer, A6 comprises a dendrimer with the same number of labels and 20 to 30 attached aptamers. As will be seen in the examples below, certain dendrimer conformations appear to consistently provide greater enhancement both in sensitivity of detection and signal amplification than others, even when used in combination with different aptamers. Surprisingly, the dendrimers that confer the greatest amount of signal amplification are not necessarily those with the highest number of labels. Thus, constructing an aptamer-dendrimer conjugates that improves signal amplification may involves further structural considerations.

### Example 2

### Enhanced Sensitivity of Detection by Aptamer-Dendrimer Conjugates of the a Series in Comparison to Aptamer Alone Upon Direct Detection of Thrombin Analyte Absorbed Onto Microliter Plates at a Higher Concentration

The ability of aptamer-dendrimer conjugates (listed in Table 1 hereinabove) to increase the sensitivity of detection relative to aptamer alone was also examined using a higher concentration of aptamer and aptamer-dendrimer conjugates than those used above, a larger amount of attached thrombin target and a longer incubation period of the aptamers or aptamer-dendrimer conjugates with the thrombin target.

To accomplish this, Aptamer A, which recognizes thrombin, was biotinylated to allow for detection of aptamer binding. Binding and signal production of the aptamer A0 was compared to binding by the same unbiotinylated aptamer conjugated with each of seven different biotinylated dendrimer conformations (numbers A1-A7 in the series above). These dendrimer conformations were shown in Table 2 above and they include conjugates with 1, 5, or 20-30 aptamers complexed per dendrimer, and with 50, 120, 350 or 900 biotin labels.

Thrombin was fixed to microtiter wells by exposing wells to 100 μl of solutions containing 500 ng of thrombin/100 μl. For comparison of their ability to detect the absorbed thrombin, the biotinylated aptamer A and the seven different biotinylated aptamer-dendrimer conjugates shown in the table below were applied (100 ng/well) to duplicate wells that had been exposed to each thrombin concentration. Following a three-hour incubation, the wells containing attached biotinylated aptamer and biotinylated aptamer-dendrimer complexes were washed, exposed first to streptavidin-HRP and then to TMB substrate. The reaction was stopped with TMB stop solution and read at OD 420 nm.

Following detection of signal, the signal to background ratio was calculated for each aptamer or aptamer-dendrimer conjugate by comparing to the signal observed in wells with thrombin to the signal obtained in the control wells with no thrombin. The results are shown below in Table 4.

TABLE 4
Comparison of thrombin detection by Aptamer A alone (A0)
and by aptamer-dendrimer conjugates of the A series (A1-
A7), shown as signal:background ratio in comparison with
control wells containing no thrombin using higher concentrations
of aptamers and aptamer-dendrimer conjugates, and thrombin
target and longer incubation times

| Aptamer/conjugate | 500 ng |
|---|---|
| A0 | 1.13 |
| A1 | 1.34 |
| A2 | 1.38 |
| A3 | 1.25 |
| A4 | 1.5 |
| A5 | 1.28 |
| A6 | 2.4 |
| A7 | 1.35 |

From Table 4 it can be seen that under these conditions, A0, the aptamer alone, gives a signal to background ratio with 500 ng of thrombin that is comparable to what was observed in the previous example with all of the aptamer-dendrimer conjugates at 100 ng of thrombin and some of them at 25 ng of thrombin. Since in this study aptamer A0 was applied at twice the concentration and with a longer exposure time, these observations offer additional evidence that almost all the aptamer-dendrimer conjugates of the A series (all those which detected 100 ng of thrombin) are at least 5 times more sensitive than the aptamer alone and some of them (which detected 25 ng of thrombin) are at least 20 times as sensitive as the aptamer alone when it is not conjugated to any dendrimer. The actual increase in sensitivity is likely to be higher because of the higher concentration of aptamer and longer incubation time used in this presently described Example in comparison to the conditions used in Example 1 above. It is thus demonstrated that the addition of the dendrimers in all the aptamer-dendrimer conjugates of the A series offers an increase in sensitivity of target detection and that this increase in sensitivity is greater than an order of magnitude in the case of some of the aptamer-dendrimer conjugates.

The average signal to background ratio of all the aptamer-dendrimer conjugates is 1.50 compared to 1.13 for the aptamer alone. As in Example 1, the conjugate with 900 labels was not the one which gave the greatest signal amplification.

While most of the aptamer-dendrimer conjugates give a similar level of signal enhancement of 1.25 to 1.5, conjugate A6, comprised of a 4 layer dendrimer with 20-30 aptamers attached and 350 labels, had a distinctly higher level of signal to background. In the case of aptamer-dendrimer conjugate A6, the signal to background ratio reached 2.4 fold. Thus, aptamer-dendrimer conjugate A6, which displayed an increase in sensitivity to target of 20 to 40 fold relative to aptamer alone, also had a signal to background ratio more than two fold greater than the aptamer alone.

### Example 3

### Aptamer-Dendrimer Conjugates of the B Series (B1-B7) Provide a Greatly Enhanced Signal to Background Ratio Relative to Aptamer B Alone (B0)

A study was carried out as above in Example 1, but employing the B series of aptamer and aptamer-dendrimer conjugates, as shown in the Table 1 above and Table 5 below, and applying these to wells over a wide analyte concentration, namely, wells exposed to 62.5, 125, 250, 500 and 1000 ng of thrombin.

TABLE 5
Aptamer-dendrimerconjugates of the B series

| Code | Aptamer | Aptamers per Dendrimer | Labels |
|---|---|---|---|
| B0 | Aptamer B | No dendrimer | 1 label |
| B1 | Aptamer B | 1 | 50 labels |
| B2 | Aptamer B | 1 | 120 labels |
| B3 | Aptamer B | 1 | 350 labels |
| B4 | Aptamer B | 5 | 50 labels |
| B5 | Aptamer B | 5 | 120 labels |
| B6 | Aptamer B | 20-30 | 350 labels |
| B7 | Aptamer B | 20-30 | 900 labels |

As can be seen in FIG. 1, detection of thrombin, defined in this instance as a signal to background ratio greater than 1.5, is observed beginning from 500 ng. At this thrombin amount, the aptamer alone, B0, displays a signal to background ratio of 1.57. In contrast, two of the aptamer-dendrimer conjugates, B1 and B6, display signal to background ratios greater than 2 (4.04 and 2.65), respectively.

At 1000 ng of thrombin, the signal to background ratios of aptamer-dendrimer conjugates are even more enhanced relative to that for the aptamer alone. While the aptamer alone (B0) continues to display a signal to background ratio below 2 (1.65), four of the aptamer-dendrimer conjugates display signal to background ratios above 2. More specifically, the ratio for aptamer-dendrimer conjugate B1 is 4.21; for conjugate B4 it is 13.22, for conjugate B6 it is 13.19 and for conjugate B7 it is 3.92.

As in the study above, the aptamer-dendrimer conjugate with the largest number of labels (B7 with 900 labels) is not the one that provides the greatest signal amplification. This again suggests that other factors, such as steric factors, in addition to the number of labels and bound aptamers per dendrimer, also determine the ability of aptamer-dendrimer conjugates to increase the sensitivity of detection.

The conjugates that provide the greatest signal enhancement—achievement of signal to background ratios of more than 13 as compared to less than 2 for the aptamer alone—are B4, with 5 aptamers conjugated to each dendrimer and 50 biotin labels, and B6 with 20-30 aptamers conjugated to each dendrimer and 350 labels. In Examples 1 and 2, a structurally comparable aptamer-dendrimer conjugate from the A series, A6, also gave the greatest sensitivity and signal amplification as compared to all the other aptamer-dendrimer conjugates in the series. It is seen that, unlike what might have been expected, the highest-labeled conjugates are not those which provide the greatest signal enhancement. Rather, particular dendrimer conformations appear to provide the greatest enhancement in sensitivity and signal amplification for a range of different aptamers. Thus, for each aptamer of commercial interest it will be possible to identify particular aptamer-dendrimer conjugates that will significantly improve its performance in detection, trapping, etc. by examining a small number of potential aptamer-dendrimer conjugates as demonstrated here.

### Example 4

### Significantly Higher Signal to Background Ratios are Achieved at Higher Concentrations of Aptamer-Dendrimer Conjugates of the B Series (B1-B 7)

A study was carried out to determine if higher concentrations and/or longer binding times would further improve target detection sensitivity, enabling detection of lower thrombin amounts and even higher signal to background ratios relative to that observed with aptamer alone, The study was carried out as in Example 3, but in the presence of 100 ng/well aptamer/dendrimer conjugates of the B series (Table 5) and a with a 3 hour incubation. A narrower analyte concentration was used in this case, with wells exposed to 62.5, 125 and 250 ng of thrombin.

The results shown in FIG. 2 demonstrate that under these conditions, thrombin at a low amount of 250 ng and even of 125 ng was detected and the signal to background ratios achieved was higher. At 125 ng of thrombin, the signal to background ratio of the aptamer alone was 6.30, similar to that of most of the other tested aptamer/dendrimer conjugates. A much higher signal to background ratio, of 16.57, was displayed at this concentration by the B6 conjugate.

Significantly, at 250 ng of thrombin, the signal to background ratios of three of the conjugates, B1, B4 and B6, was 20 or higher. As indicated above in Example 3, these conjugates also displayed the highest signal to background ratios when they were tested at a lower concentration. Conjugate B1, composed of one aptamer conjugated to each two layers dendrimer and 50 biotin labels, displayed a signal to background ratio of 20.11, conjugate B4, composed of five aptamers conjugated to each dendrimer and 50 biotin labels, displayed a ratio of 25.12 and conjugate B6, composed of 20-30 aptamers conjugated to each four layers dendrimer and 350 labels, displayed a ratio of 47.96. These conjugates all give a signal that is more than 50 fold enhanced over the signal of the aptamer alone, namely, 60, 76 and 145 fold higher for B1, B4 and B6, respectively. Furthermore, in the present case, as in all the previous Examples, the amplification fold, as calculated on a weight basis (100 ng of aptamer alone or aptamer/dendrimer conjugate per well), is actually 1.5 to 2.5 orders of magnitude lower than it would be if the calculation was performed on a molar basis, due to the much higher molecular weight of the conjugates, as seen in Table 1.

Finally, the shape of the curves obtained with the aptamer/dendrimer conjugates demonstrate that quantitative information can be derived in detection studies using these recognition elements. All the conjugates B1, B4, B6 and B7 display a significantly higher signal in the presence of 250 ng of thrombin than in the presence of 125 ng. In the case of B1 and B6 this is about 2.5× greater, in the case of B4 about 4× greater, and, in the case of B7, the size of the signal in the presence of 250 ng of target is about 2× that of the size in the presence of 125 ng of target, indicating a clear is linear relationship. These results indicate that aptamer/dendrimer conjugates can be useful in quantification of target analytes.

### Example 5

### A Multiply Biotinylated Aptamer Dendrimer Conjugate Increases Efficiency of Analyte Trapping

A multiply biotinylated aptamer-dendrimer conjugate was found to be more efficient in analyte trapping than biotinylated aptamer alone, as judged by the amount of the target analyte remaining in the solution following pelleting with Neutravidin Agarose Resin.

The capability of biotinylated DNA-aptamer-dendrimer conjugates in enhancing target trapping was examined using a biotin-avidin pull down assay, as described in the “Experimental procedures” section, above.

Table 6 below shows the OD values obtained for the supernatant solutions containing unbound thrombin, which were obtained in the pull down assay performed with various aptamer-dendrimer conjugates of the A series (A1, A4 and A5), using High Capacity NeutrAvidin Agarose Resin. In this analysis, a smaller amount of thrombin in the supernatant is indicative of the greater trapping/partitioning capacity (or ability) of the target recognition composition. Calculating the relative ability of the different reagents to deplete thrombin from the solution is based on comparing the results presented in Table 6 to the calibration curve prepared for thrombin, in which thrombin concentration of 0, 200, 300, 400 and 500 ng resulted in O.D. (450 nm) values of 0.269, 0.646, 0.829, 1.028 and 1.19, respectively.

TABLE 6
Relative efficiencies of thrombin trapping by Aptamer A alone
(A0) and by aptamer-dendrimer conjugates of the A series (A1,
A4 and A5) pelleted with High Capacity NeutrAvidin Agarose Resin,
shown as OD of the supernatant solution containing unbound thrombin
and compared with ODs of a calibration curve

| Trapping agent | OD |
|---|---|
| no aptamer | 1.27 |
| A0 | 1.12 |
| A1 | 1.23 |
| A4 | 1.31 |
| A5 | 0.64 |

By comparing the results shown in Table 6 above with the calibration curve, in the absence of any aptamer, the estimated amount of thrombin remaining in the supernatant, following pelleting with the Neutravidin Agarose Resin, is 500 ng, which is the amount that was originally distributed in the different samples. This is also the case for the samples exposed to biotinylated aptamer alone (A0) and for two of the aptamer/dendrimer conjugates tested (A1 and A4). In contrast, the amount of unbound thrombin remaining in the supernatant following exposure to the A5 aptamer/dendrimer conjugate was significantly reduced. The amount remaining in the supernatant was about 200 ng, as judged by comparing the OD obtained for A5 (0.64) with that obtained for an amount of 200 ng thrombin in the calibration curve (0.646). It should be noted that this conjugate comprises a two layers dendrimer attached to five aptamers and labeled with 120 biotin labels.

These results demonstrate the increased analyte trapping and partitioning efficiency of some aptamer/dendrimer conjugates, multiply labeled with biotin as partitioning moieties, as compared to the aptamer alone, with a single biotin moiety.

Moreover, these results demonstrate the feasibility of using the target-recognition composition of the invention for trapping and thereby removing an undesired target compound from a particular substance.

### Example 6

### Aptamer-Dendrimer Conjugates Increases Efficiency of Analyte Trapping—a Gel Analysis

The increased efficiency of analyte trapping of the aptamer-dendrimer conjugates was also demonstrated by gel analysis of the target analyte released from Neutravidin Agarose Resin.

The trapping ability of a biotinylated aptamer that recognizes thrombin (A0) was further compared to the trapping ability of the same aptamer conjugated with different multiply biotinylated dendrimers (A1, A4, A5 and A6) by performing a gel analysis of the target analyte released from Neutravidin Agarose Resin. The aptamer or each of these multiply biotinylated dendrimers (target recognition compositions) were incubated with thrombin, as described herein above (Example 5). The amount of trapped thrombin was determined by pelleting thrombin trapped by biotinylated aptamers or aptamer/dendrimer conjugates with Neutravidin Agarose Resin, release of thrombin into sample buffer containing DTT and performing SDS-gel analysis followed by silver staining.

As shown in FIG. 3, thrombin alone displays a background doublet at the anticipated MW 36 KDa (the doublet may reflect the presence of a degradation product of alpha thrombin known as beta thrombin). There is some increase in the intensity of the thrombin band in the presence of the aptamer alone (A0), which is indicative of trapping (partitioning). Apparently, the level of thrombin trapping is not increased in the presence of the aptamer/dendrimer conjugates A1 and A4. In contrast, there is enhanced binding of thrombin to the NeutrAvidin Agarose Resin following incubation of thrombin with the aptamer dendrimer conjugates A5 and A6. Thus, these aptamer-dendrimer conjugates are more efficient in trapping (partitioning) thrombin than the aptamer per se (A0).

These results clearly demonstrate that trapping using the target recognition composition of the invention may be used for enriching a target compound and thereby for purification purpose.

1. A method for trapping, removing, detecting or quantitating, at least one target compound in or from a substance or any preparation or sample thereof, comprising the steps of:
a. providing at least one target-recognition composition comprising at least one nucleic acid-based aptamer that specifically recognizes and binds said at least one target compound, or complex thereof, or any parts or fragments thereof, or any combinations thereof, and at least one nucleic acid based dendrimer labeled with at least one trapping, partitioning or detectable moiety;
b. contacting said substance, or any preparation, sample, or an aliquot thereof with at least one of said target-recognition composition of (a) under conditions suitable for recognition and binding of said at least one target compound or any parts or fragments thereof, or any combinations thereof, by said at least one nucleic acid based aptamer to form at least one target-aptamer complex; and
c. trapping or detecting said at least one trapping or detectable moiety in said labeled dendrimer comprised within said target-recognition composition, which dendrimer is bound to or conjugated with said at least one aptamer in said target-aptamer complex, thereby trapping, removing or determining the presence and the amount of said target compound in or from said substance.

2. The method according to claim 1 for trapping or removing at least one target compound from a substance or any preparation or sample thereof, comprising the steps of:
a. providing at least one target-recognition composition comprising at least one nucleic acid-based aptamer that specifically recognizes and binds said at least one target compound, or complex thereof, or any parts or fragments thereof, or any combinations thereof, and at least one nucleic acid based dendrimer labeled with at least one trapping or partitioning;
b. contacting said substance, or any preparation, sample, or an aliquot thereof with at least one of said target-recognition composition of (a) under conditions suitable for recognition and binding of said at least one target compound or any parts or fragments thereof, or any combinations thereof, by said at least one nucleic acid based aptamer to form at least one target-aptamer complex; and
c. trapping said at least one trapping moiety in said labeled dendrimer comprised within said target-recognition composition, which dendrimer is bound to or conjugated with said at least one aptamer in said target-aptamer complex, thereby trapping or removing said target compound in or from said substance.

3. The method according to claim 1, for detecting or quantitating, at least one target compound in or from a substance or any preparation or sample thereof, comprising the steps of:
a. providing at least one target-recognition composition comprising at least one nucleic acid-based aptamer that specifically recognizes and binds said at least one target compound, or complex thereof, or any parts or fragments thereof, or any combinations thereof, and at least one nucleic acid based dendrimer labeled with at least one detectable moiety;
b. contacting said substance, or any preparation, sample, or an aliquot thereof with at least one of said target-recognition composition of (a) under conditions suitable for recognition and binding of said at least one target compound or any parts or fragments thereof, or any combinations thereof, by said at least one nucleic acid based aptamer to form at least one target-aptamer complex; and
c. detecting said at least one detectable moiety in said labeled dendrimer comprised within said target-recognition composition, which dendrimer is bound to or conjugated with said at least one aptamer in said target-aptamer complex, thereby determining the presence and the amount of said target compound in said substance.

4. The method according to claim 1 further comprising the steps of:
(i) providing at least one capture-composition comprising at least one nucleic acid based or amino-acid-based recognition element that specifically recognizes and binds at least one of said target compound or any parts or fragments thereof, or any combinations thereof, said at least one capture-composition is attached to a solid support or a partitioning element; and
(ii) contacting said substance or any preparation or sample thereof with at least one of said capture-composition of (i) under conditions suitable for recognition and binding of said at least one target compound or any parts or fragments thereof, or any combinations or complex thereof, by said at least one nucleic acid based or amino-acid-based recognition element comprised within said capture-composition to form at least one complex comprising the target compound or any complex thereof and the capture-composition attached to said solid support or partitioning element.

5. The method according to claim 4, comprising the steps of:
a. providing at least one target-recognition composition comprising at least one nucleic acid based aptamer that specifically recognizes and binds said at least one target compound, or complexes thereof, or any parts or fragments thereof, or any combinations thereof, and at least one nucleic acid based dendrimer labeled with at least one detectable moiety;
b. providing at least one capture-composition comprising at least one nucleic acid based or amino-acid-based recognition element that specifically recognizes and binds at least one of said target compound or any parts or fragments thereof, or any complexes or any combinations thereof, said at least one capture-composition is attached to a solid support or a partitioning element;
c. contacting said substance or any preparation or sample thereof with at least one of said capture-composition of (b) under conditions suitable for recognition and binding of said at least one target compound or any parts or fragments thereof, or any combinations thereof, by said at least one nucleic acid based or amino-acid-based recognition element comprised within said capture-composition to form at least one target-capture-composition complex attached to said solid support or partitioning element;
d. contacting said at least one target-capture-composition complex obtained in step (c) with at least one of said target-recognition composition of (a) under conditions suitable for recognition and binding of said at least one target compound, or complex or composition thereof, in said target-capture-composition complex by said at least one nucleic acid-based aptamer comprised within said target-recognition composition to form at least one target recognition composition-target compound-capture-composition complex attached to said solid support or partitioning element; and
e. detecting said at least one detectable moiety in said labeled dendrimer comprised within said target-recognition composition in said target recognition composition-target compound-capture-composition complex attached to said solid support or partitioning element; wherein at least one of detection and determination of the amount of said at least one detectable moiety indicates at least one of the presence and quantity of said target compound in said substance or any preparation or sample thereof.

6. The method according to claim 4, comprising the steps of:
a. providing at least one target-recognition composition comprising at least one nucleic acid based aptamer that specifically recognizes and binds said at least one target compound, or complexes thereof, or any parts or fragments thereof, or any combinations thereof, and at least one nucleic acid based dendrimer labeled with at least one detectable moiety;
b. providing at least one capture-composition comprising at least one nucleic acid based or amino-acid-based recognition element that specifically recognizes and binds at least one of said target compound or any parts or fragments thereof, or any complexes or any combinations thereof, said at least one capture-composition is attached to a solid support or a partitioning element;
c. contacting said substance or any preparation or sample thereof with at least one of said target-recognition composition of (a) under conditions suitable for recognition and binding of said at least one target compound or any parts or fragments thereof, or any combinations thereof, by said at least one nucleic acid based aptamer to form at least one complex of target compound with said target-recognition composition;
d. contacting said at least one target compound-target-recognition composition complex obtained in step (c) with at least one of said capture-composition of (b) under conditions suitable for recognition and binding of said at least one target compound, or complex or combination thereof, in said target compound-target recognition-composition complex by said at least one nucleic acid based or amino-acid-based recognition element comprised within said capture-composition to form at least one target recognition composition-target compound-capture-composition complex attached to said solid support or partitioning element; and
e. detecting said at least one detectable moiety in said labeled dendrimer comprised within said target-recognition composition, that is bound to or conjugated with said at least one aptamer in said target recognition composition-target compound-capture-composition complex attached to said solid support or partitioning element; wherein at least one of detection and determination of the amount of said at least one detectable moiety indicates at least one of the presence and quantity of said target compound in said substance or any preparation or sample thereof.

7. The method according to claim 4, wherein said nucleic acid based or amino-acid-based recognition element comprised within said capture-composition is any one of a nucleic acid selected from: aptamer, probe, primer, or oligonucleotide specific for said target, or an amino-acid selected from: an antibody, peptide, ligand, receptor or substrate, or any small molecule specific for said target.

8. The method according to claim 1, wherein said aptamer and dendrimer comprised in said target-recognition composition are attached or conjugated prior to contacting said composition with said substance or any sample thereof.

9. The method according to claim 1, wherein said aptamer and dendrimer comprised in said target-recognition-composition are attached or conjugated after contacting said composition with said substance or any sample thereof.

10. The method according to claim 1, wherein said dendrimers are comprised of deoxyribonucleic acids.

11. The method according to claim 10, wherein said dendrimer comprises between about 1 to about 5 layers and between 100 to 500 labeling moieties.

12. The method according to claim 1, wherein said dendrimers are each attached to or conjugated with more than one identical or different aptamers.

13. The method according to claim 12, wherein said dendrimers are each attached to or conjugated with more than one identical aptamers.

14. The method according to claim 12, wherein said aptamers are different aptamers recognizing different or identical targets.

15. The method according to claim 14, wherein said aptamers are different aptamers that recognize identical targets.

16. The method according to claim 14, wherein said aptamers are different aptamers that recognize different targets.

17. The method according to claim 12, wherein said target recognition composition comprises between about 1 to 50 aptamers.

18. A kit for trapping, removing, detecting, or quantitating, at least one target compound in/or from a substance or any preparation or sample thereof, comprising:
a. at least one nucleic acid based aptamer that specifically recognizes and binds said at least one target compound, or complex thereof, or any parts or fragments thereof, or any combinations thereof; and
b. at least one nucleic acid based dendrimer labeled with at least one trapping, partitioning or detectable moiety;
c. optionally, instructions for carrying out the detection or trapping of said at least one detectable or trapping moiety in said labeled dendrimer.

19. The kit according to claim 18, for detecting or quantitating at least one target compound in a substance or any sample thereof, wherein said kit further comprises:
d. at least one capture-composition comprising at least one nucleic acid based or amino acid-based recognition element that specifically recognizes and binds at least one of said target compound or any parts or fragments thereof, or any complex or combinations thereof, said at least one capture-composition is attached to a solid support or a partitioning element;
e. optionally, at least one compartment containing at least one said capture-composition of (d) and optionally an array, wherein each of said capture-composition is located in a defined and predetermined position in said array; and
f. optionally, instructions for carrying out the detection or trapping of said at least one detectable or trapping moiety in said labeled dendrimer that is bound to said at least one aptamer in an aptamer-target-capture-composition complex attached to a solid support or a partitioning element.

20. A device for trapping, removing, detecting, or quantitating at least one target compound in/or from a substance or any preparation or sample thereof, comprising:
a. at least one target-recognition composition comprising at least one nucleic acid based aptamer that specifically recognizes and binds said at least one target compound, or complex thereof, or any parts or fragments thereof, or any combinations thereof, and at least one nucleic acid based dendrimer labeled with at least one trapping, partitioning or detectable moiety; and
b. a solid support suitable for the reception and transport of said sample.

21. The device according to claim 20, wherein said device is a lateral flow device for detecting or quantitating at least one target compound in a substance or any preparation or sample thereof, said device comprises:
a. a solid support suitable for the reception and transport of said sample;
b. at least one target-recognition composition comprising at least one nucleic acid based aptamer that specifically recognizes and binds said at least one target compound, or complex thereof, or any parts or fragments thereof, or any combinations thereof, and at least one nucleic acid based dendrimer labeled with at least one detectable moiety, said at least one target-recognition composition is located in a predetermined specific initiation zone in said solid support; and
c. at least one capture-composition comprising at least one nucleic acid based or amino acid-based recognition element that specifically recognizes and binds at least one of said target compound or any complex thereof, said at least one capture-composition is attached to said solid support in a predetermined location in an termination zone in said solid support.

22. The device according to claim 20, wherein said device is a flow-through device for trapping or removing at least one target compound from a substance or any preparation or sample thereof, said device comprises:
a. a solid support suitable for the reception and transport of said sample comprising a sample port and a flow path extending from the sample port, wherein said flow path includes partitioning-elements for capturing said at least one trapping or partitioning moiety attached to said dendrimer comprised within said target-recognition composition;
b. at least one target-recognition composition provided with the sample port or captured in said flow path by said partitioning-elements.


SEQUENCE LISTING (5 sequences)

SEQ ID NO: 1 - DNA, 15 bp, ARTIFICIAL
  APTAMER A
ggttggtgtggttgg

SEQ ID NO: 2 - DNA, 27 bp, ARTIFICIAL
  APTAMER B
gtccgtggtagggcaggttggggtgac

SEQ ID NO: 3 - DNA, 20 bp, ARTIFICIAL
  Bridging molecule
tcacatacgactccgaaaaa

SEQ ID NO: 4 - DNA, 22 bp, artificial
  Aptamer A with a 3′ linker
ggttggtgtggttggtttttcg

SEQ ID NO: 5 - DNA, 34 bp, artificial
  Aptamer B with a 3′ linker
gtccgtggtagggcaggttggggtgactttttcg